(19) United States
(12) Patent Application Publication
(10) Pub. No.: US 2012/0149710 A1
(43) Pub. Date: Jun. 14, 2012

(54) SORTASE A INHIBITORS

(75) Inventors: Michael E. Jung, Los Angeles, CA (US); Robert T. Clubb, Culver City, CA (US); Sung Wook Yi, Los Angeles, CA (US); Nutee Suree, Lampang (TH); Jeremy Justin Clemens, San Diego, CA (US)

(73) Assignee: The Regents of the University of California

(21) Appl. No.: 13/391,574

(22) PCT Filed: Aug. 23, 2010
(86) PCT No.: PCT/US10/46394

§ 371 Date: Feb. 21, 2012

Related U.S. Application Data
(60) Provisional application No. 61/236,453, filed on Aug. 24, 2009.

(51) Int. Cl. A61K 31/50; C07D 401/12; C07D 403/12; C07D 417/06; A61P 31/04; C07D 231/18; A61K 31/501; A61K 31/427; A61K 31/426; A61K 31/415; C07D 237/18; C07D 277/36

(52) U.S. Cl. 514/252.02; 544/240; 544/238; 548/200; 548/183; 548/370.1; 514/247; 514/252.03; 514/365; 514/369; 514/404

(57) ABSTRACT

Bacterial infections, including Methicillin resistant Staphylococcus aureus (MRSA) infections are a major health problem that has created a pressing need for new antibiotics. Pyridazinone, rhodanine, and pyrazolethione compounds effective inhibit the enzymatic activity of sortase A (srtA) found in gram positive bacteria are disclosed. A structure activity relationship (SAR) analysis led to the identification of several pyridazinone and pyrazolethione analogs that inhibit SrtA with IC₅₀ values in the sub-micromolar range. Compounds that inhibit the S. aureus SrtA sortase may function as potent anti-infective agents as this enzyme attaches virulence factors to the cell wall. Many of these molecules also inhibit the sortase enzyme from B. anthracis suggesting that they may be generalized sortase inhibitors.

The novel compounds, compositions, uses, formulations, medicaments, articles of manufacture provide improved materials, uses, and treatments useful in combating infectious disorders.

### STATEMENT OF GOVERNMENT SUPPORT

This invention was made with Government support of Grant Nos. AI052217 awarded by the National Institutes of Health. The U.S. government has certain rights in this invention.

### FIELD OF USE

This application discloses compounds, compositions, uses, medicaments, and methods related to sortase A and other bacterial enzymes, binding to and inhibition of sortase A and other bacterial enzymes, the use of such compounds and compositions, the preparation of medicaments comprising such compounds and compositions, and treatments of bacterial infections and disorders related to sortase A and other bacterial enzymes, and related subject matter.

### BACKGROUND

The rise of community- and hospital-acquired methicillin resistant Staphylococcus aureus (MRSA) is a major health problem that has created a pressing need for new antibiotics (Talbot, G. H.; Bradley, J.; Edwards, J. E., Jr.; Gilbert, D.; Scheld, M.; Bartlett, J. G. Clin. Infect. Dis. 2006, 42, 657). More than 90,000 Americans acquire potentially deadly MRSA infections each year, which annually are estimated to kill more people than AIDS in the United States (Klevens, R. M.; Morrison, M. A.; Nadle, J.; Petit, S.; Gershman, K.; Ray, S.; Harrison, L. H.; Lynfield, R.; Dumyati, G.; Townes, J. M.; Craig, A. S.; Zell, E. R.; Fosheim, G. E.; McDougal, L. K.; Carey, R. B.; Fridkin, S. K. JAMA 2007, 298, 1763). Proteins displayed on the surface of S. aureus play key roles in the infection process as they promote bacterial adhesion to host cells and tissue, acquire essential nutrients and circumvent the immune response (Navarre, W. W.; Schneewind, O. Microbiol. Mol. Biol. Rev. 1999, 63, 174). Most surface proteins in S. aureus are attached to the cell wall by the Sortase A (SrtA) enzyme (Marraffini, L. A.; Dedent, A. C.; Schneewind, O. Microbiol. Mol. Biol. Rev. 2006, 70, 192; Paterson, G. K.; Mitchell, T. J. Trends Microbiol. 2004, 12, 89; Ton-That, H.; Marraffini, L. A.; Schneewind, O. Biochim. Biophys. Acta 2004, 1694, 269; Mazmanian, S. K.; Liu, G.; Hung, T. T.; Schneewind, O. Science 1999, 285, 760; Ton-That, H.; Liu, G.; Mazmanian, S. K.; Faull, K. F.; Schneewind, O. Proc. Natl. Acad. Sci. USA 1999, 96, 12424). SrtA is located on the extracellular surface and catalyzes a transpeptidation reaction that joins an LPXTG sorting signal within the surface protein precursor to the cell wall precursor molecule lipid-II [undecaprenyl-pyrophosphate-MurNAc(-L-Ala-D-iGln-L-Lys(NH₂-Gly₅)-D-Ala-D-Ala)-β1-4-GlcNAc)] (Mazmanian, S. K.; Liu, G.; Hung, T. T.; Schneewind, O. Science 1999, 285, 760; Ton-That, H.; Liu, G.; Mazmanian, S. K.; Faull, K. F.; Schneewind, O. Proc. Natl. Acad. Sci. USA 1999, 96, 12424; Schneewind, O.; Model, P.; Fischetti, V. A. Cell 1992, 70, 267; Schneewind, O.; Mihaylovapetkov, D.; Model, P. EMBO J. 1993, 12, 4803). The lipid-II linked protein product is then incorporated into the cell wall by the transglycolysation and transpeptidation reactions of cell wall synthesis (Perry, A. M.; Ton-That, H.; Mazmanian, S. K.; Schneewind, O. J. Biol. Chem. 2002, 277, 16241; Ruzin, A.; Severin, A.; Ritacco, F.; Tabei, K.; Singh, G.; Bradford, P. A.; Siegel, M. M.; Projan, S. J.; and Shlaes, D. M.; J. Bacteriol. 2002, 184, 2141; Schneewind, O.; Fowler, A.; Faull, K. F. Science 1995, 268, 103). Small molecules that inhibit the SrtA transpeptidation reaction may be powerful anti-infective agents as srtA⁻ strains of S. aureus fail to display many virulence factors and exhibit reduced virulence (Zink, S. D.; Burns, D. L. Infect. Immun. 2005, 73, 5222; Weiss, W. J.; Lenoy, E.; Murphy, T.; Tardio, L.; Burgio, P.; Projan, S. J.; Schneewind, O.; Alksne, L. J. Antimicrob. Chemother. 2004, 53, 480; Jonsson, I. M.; Mazmanian, S. K.; Schneewind, O.; Verdrengh, M.; Bremell, T.; Tarkowski, A. J. Infect. Dis. 2002, 185, 1417; Mazmanian, S. K.; Liu, G.; Jensen, E. R.; Lenoy, E.; Schneewind, O.; Proc. Natl. Acad. Sci. USA 2000, 97, 5510; Mazmanian, S. K.; Ton-That, H.; Su, K.; Schneewind, O. Proc. Natl. Acad. Sci. USA 2002, 99, 2293; Bierne, H.; Mazmanian, S. K.; Trost, M.; Pucciarelli, M. G.; Liu, G.; Dehoux, P.; Jansch, L.; Garcia-del Portillo, F.; Schneewind, O.; Cossart, P. Mol. Microbiol. 2002, 43, 869; Garandeau, C.; Reglier-Poupet, H.; Dubail, L.; Beretti, J. L.; Berche, P.; Charbit, A. Infect. Immun. 2002, 70, 1382; Kharat, A. S.; Tomasz, A. Infect. Immun. 2003, 71, 2758; Chen, S.; Paterson, G. K.; Tong, H. H.; Mitchell, T. J.; Demaria, T. F. FEMS Microbiol. Lett. 2005, 253, 151; Paterson, G. K.; Mitchell, T. J. Microbes Infect. 2005, 12, 89; Bolken, T. C.; Franke, C. A.; Jones, K. F.; Zeller, G. O.; Jones, C. H.; Dutton, E. K.; Hruby, D. E. Infect. Immun. 2001, 69, 75). There are several antibiotics that are effective at treating Staphylococcus aureus and other bacterial infections. SrtA inhibitors may also be useful in treating infections caused by other Gram-positive pathogens, since many also use related enzymes to attach virulence factors to the cell wall and to assemble pili that promote bacterial adhesion (Scott, J. R.; Zahner, D. Mol. Microbiol. 2006, 62, 320; Mandlik, A.; Swierczynski, A.; Das, A.; Ton-That, H. Trends Microbiol. 2008, 16, 33). Sortases can be classified into five distinct families based on their primary sequence (Comfort, D.; Clubb, R. T. Infect. Immunol. 2004, 72, 2710). Enzymes most closely related to the S. aureus SrtA protein appear to be the best candidates for inhibitor development as their elimination in other bacterial pathogens attenuates virulence (e.g. Listeria monocytogenes, Streptococcus pyogenes and Streptococcus pneumoniae (Maresso et al., Pharmacol. Rev. 2008, 60, 128; Suree et al., Mini-Rev. Med. Chem. 2007, 7, 991). Finally, SrtA is not required for the growth of S. aureus in cell cultures. Therefore, anti-infective agents that work by inhibiting SrtA could have a distinct advantage over conventional antibiotics as they may be less likely to induce selective pressure that leads to drug resistance (Mazmanian, S. K.; Liu, G.; Hung, T. T.; Schneewind, O. Science 1999, 285, 760; Cossart, P.; Jonquieres, R. Proc. Natl. Acad. Sci. USA 2000, 97, 5013).

A number of different strategies have been employed to search for sortase inhibitors (reviewed in refs. 28,29,31). These include screening natural products (Kim, S. H.; Shin, D. S.; Oh, M. N.; Chung, S. C.; Lee, J. S.; Chang, I. M.; Oh, K. B. Biosci. Biotechnol. Biochem. 2003, 67, 2477; Kim, S. H.; Shin, D. S.; Oh, M. N.; Chung, S. C.; Lee, J. S.; Oh, K. B. Biosci. Biotechnol. Biochem. 2004, 68, 421; Kim, S. W.; Chang, I. M.; Oh, K. B. Biosci. Biotechnol. Biochem. 2002, 66, 2751; Oh, K. B., Mar, W., Kim, S., Kim, J. Y., Oh, M. N., Kim, J. G., Shin, D., Sim, C. J.; Shin, J. Bioorg. Med. Chem. Lett. 2005, 15, 4927; Jang, K. H.; Chung, S. C.; Shin, J.; Lee, S. H.; Kim, T. I.; Lee, H. S.; Oh, K. B. Bioorg. Med. Chem. Lett. 2007, 17, 5366; Kang, S. S.; Kim, J. G.; Lee, T. H.; Oh, K. B. Biol. Pharm. Bull. 2006, 29, 1751; Park, B. S.; Kim, J. G.; Kim, M. R.; Lee, S. E.; Takeoka, G. R.; Oh, K. B.; Kim, J. H. J. Agric. Food Chem. 2005, 53, 9005) and small compound libraries (Maresso, A. W.; Wu, R.; Kern, J. W.; Zhang, R.; Janik, D.; Missiakas, D. M.; Duban, M. E.; Joachimiak, A., Schneewind, O. J. Biol. Chem. 2007, 282, 23129), as well as synthesizing rationally designed peptidomimetics and small molecules (Kruger, R. G.; Barkallah, S.; Frankel, B. A.; McCafferty, D. G. Bioorg. Med. Chem. 2004, 12, 3723; Jung, M. E.; Clemens, J. J.; Suree, N.; Liew, C. K.; Pilpa, R.; Campbell, D. O.; Clubb, R. T. Bioorg. Med. Chem. Lett. 2005, 15, 5076; Liew, C. K.; Smith, B. T.; Pilpa, R.; Suree, N.; Ilangovan, U.; Connolly, K. M.; Jung, M. E.; Clubb, R. T. FEBS Lett. 2004, 571, 221; Connolly, K. M.; Smith, B. T.; Pilpa, R.; Ilangovan, U.; Jung, M. E.; Clubb, R. T. J. Biol. Chem. 2003, 278, 34061; Scott, C. J.; McDowell, A.; Martin, S. L.; Lynas, J. F.; Vandenbroeck, K.; Walker, B. Biochem. J. 2002, 366, 953). Recently, mechanism-based aryl (β-amino)ethyl ketone (AAEK) inhibitors have been reported (Maresso, A. W.; Wu, R.; Kern, J. W.; Zhang, R.; Janik, D.; Missiakas, D. M.; Duban, M. E.; Joachimiak, A., Schneewind, O. J. Biol. Chem. 2007, 282, 23129). AAEK molecules are specifically activated by sortase via a β-elimination reaction that generates an olefin intermediate that covalently modifies the active site cysteine thiol group (Maresso, A. W.; Wu, R.; Kern, J. W.; Zhang, R.; Janik, D.; Missiakas, D. M.; Duban, M. E.; Joachimiak, A., Schneewind, O. J. Biol. Chem. 2007, 282, 23129). However, these compounds only inhibit SrtA with an IC₅₀ of about 5-50 μM. (Maresso, A. W.; Wu, R.; Kern, J. W.; Zhang, R.; Janik, D.; Missiakas, D. M.; Duban, M. E.; Joachimiak, A., Schneewind, O. J. Biol. Chem. 2007, 282, 23129). Other reported compounds also need to be optimized further to be therapeutically useful as they either have limited potency, undesirable physicochemical features (e.g. high molecular weights) or inactivate the enzyme slowly (Maresso, A. W.; Schneewind, O. Pharmacol. Rev. 2008, 60, 128; Suree, N.; Jung, M. E.; Clubb, R. T. Mini-Rev. Med. Chem. 2007, 7, 991; Cossart, P.; Jonquieres, R. Proc. Natl. Acad. Sci. USA 2000, 97, 5013).

Accordingly, there is need in the art for sortase A inhibitors.

### SUMMARY

Applicants disclose herein compounds that are potent inhibitors of the sortase A (SrtA) sortase enzymes, including SrtA enzymes from S. aureus and B. anthracis. Many of these compounds inhibit the activity of these enzymes with IC₅₀ values in the high nanomolar range. Moreover, the compounds exhibit minimum inhibitory concentrations (MIC) in the millimolar range. The compounds disclosed herein are useful as anti-infective agents, for example for preventing microbial growth in the human host, while not hindering growth outside of the host.

In embodiments, the host is a human host. The compounds provide advantageous properties as compared to currently used antibiotics, for example, as they are unlikely to generate selective pressures that lead to microbial drug resistance.

To identify potent inhibitors of SrtA we performed high-throughput screening (HTS) of library containing about 30,000 compounds, which led to the identification of three promising small molecule inhibitors. These molecules can be used as the basis to develop further anti-infective agents. A structure activity relationship (SAR) analysis revealed several pyridazinone and pyrazolethione analogs that inhibit SrtA with IC₅₀ values in the sub-micromolar. These compounds are more potent than any previously described natural or synthetic inhibitor, and thus are excellent molecules for further development. Some of the subject matter disclosed herein is now found in a paper (Bioorg Med Chem. 2009, 17(20):7174-85).

Compounds disclosed herein are effective to inhibit the enzymatic activity of the SrtA sortase that is required for S. aureus infectivity. They also inhibit the activity of the SrtA sortase from Bacillus anthracis, another bacterial pathogen. Accordingly, such compounds are useful for inhibiting bacterial growth, for the preparation of medicaments for treatment of bacterial infections and disorders comprising bacteria and bacterial infections, and for the treatment of bacterial infections and related disorders.

Accordingly, disclosed herein are chemical compounds for the effective treatment of bacterial infections, especially those caused by Staphylococcus aureus. These compounds inhibit the sortase A (SrtA) protein in S. aureus and related enzymes in other bacteria. Compounds having features of the invention include three classes of compounds commonly termed pyridazinones, rhodanines and pyrazolethiones. The rhodanines are exemplified by 1, the pyridazinones are exemplified by 2-9 and the pyrazolethione compounds are exemplified by 3-12.

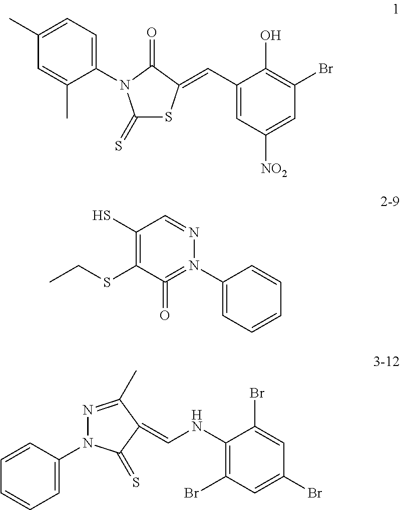

Yet a further example of a compound having features of the invention is compound 4 as shown in the following:

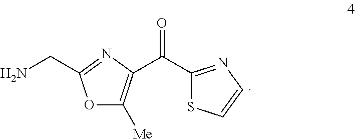

Compound 4 inhibits SrtA with an IC₅₀ of 7.2 μM. Similar compounds are also expected to act as SrtA inhibitors at similar or at even lower concentrations.

Compounds as disclosed herein, for example, molecules with a pyridazinone scaffold (such as compound 2-9 and related derivatives of the pyridazinone series) are potent sortase inhibitors. For example, four of these compounds are potent sortase inhibitors (2-58, 2-59, 2-60 and 2-61). The structures and measured inhibitory properties of these compounds are also shown in Table 4, which also provides IC₅₀ values for sortase A inhibition by these compounds. All of these compounds inhibit the SrtA sortase enzyme from Staphylococcus aureus with sub-micromolar IC₅₀ values. They are therefore the most potent sortase inhibitors that have ever been reported.

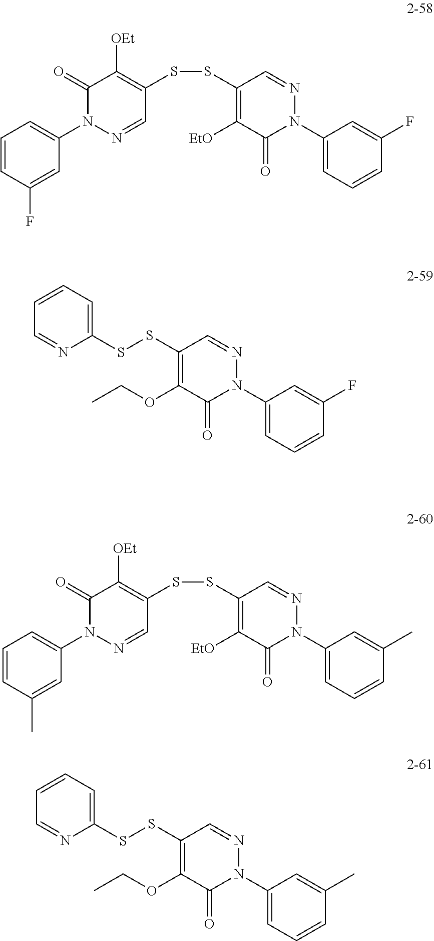

The rhodanine, pyrazolethione and pyridazinone inhibitors disclosed herein are 10 to 100 or more times more active than previously reported compounds. They reversibly inhibit the S. aureus SrtA enzyme with IC₅₀ values in the high nanomolar range. For example, molecules based on the pyridazinone frame-work can reach IC₅₀ values of about 0.20 μM or lower, as shown in Table 2. Structure-Activity Relationship (SAR) analysis has led to some of the most promising anti-infective agents as compounds 2-9 and 3-12 inhibit the enzyme with IC₅₀ values of 1.4 and 0.3 μM, respectively, and compounds 2-58, 2-59, 2-60, and 2-61 inhibit the enzyme with IC₅₀ values of 0.04, 0.01, 0.05, and 0.02 μM, respectively. Importantly, many of the molecules disclosed herein do not impair microbial growth in cell culture, suggesting that they may not spur the evolution of microbes with drug resistance. Many of these compounds also inhibit the B. anthracis SrtA, suggesting that they may be useful in treating infections caused by other species of Gram-positive bacteria in addition to S. aureus.

Methods of making these compounds are also disclosed herein.

These compounds may be used to treat a subject in need of treatment for bacterial infections. The treatments include treatment of acute bacterial infections and treatment of chronic bacterial infections. Such treatments may be prophylactic, e.g., for subjects who are in danger of acquiring such an infection (e.g., patients who are or may become immune-compromised, or who may become exposed to an infection from the environment or from a surgical procedure or hospital stay), or who are in danger of relapsing into a previous infection. Such treatments may be for bacterial infections active in the patient during the time of treatment. Such treatments may be administered after a bacterial infection, as a preventative measure to prevent recurrence of the infection.

Thus, it is disclosed herein that these compounds are suitable for treating infectious disorders, and that these compounds may be used for treating infectious disorders.

It is further disclosed herein that these compounds may be used to formulate a medicament for the treatment of an infectious disorder. Thus, the use of these compounds to formulate a medicament for treating an infectious disorder is herein disclosed.

These compounds may be included in pharmaceutical compositions. A pharmaceutical composition having features of the invention may comprise an effective amount of a compound as disclosed herein, in admixture with a pharmaceutically acceptable carrier.

Applicants further disclose methods of treating a subject in need of treatment for a bacterial infection, comprising administering an effective dose of a pharmaceutical composition comprising a compound disclosed herein. The methods of treatment include treatment of acute bacterial infections and treatment of chronic bacterial infections. The bacterial infections which may be treated include infections due to gram positive bacteria. The gram positive bacterial infections which may be treated include infections from bacteria from genera including, among others: Bacillus, Enterococcus, Lactobacillus, Lactococcus, Listeria, Staphylococcus, and Streptococcus genera. For example, the gram positive bacterial infections which may be treated include infections from bacteria selected from the group of bacteria consisting of Staphylococcus aureus (S. Aureus; SA), Listeria monocytogenes, Corynebacterium diphtheriae, Enterococcus faecalis, Clostridium perfringen, Clostridium tetani, Streptococcus pyogenes and Streptococcus pneumoniae, Bacillus anthracis (B. anthracis; BA), and other gram positive bacteria. For example, compounds disclosed herein may be used to treat infections from bacteria including Methicillin resistant Staphylococcus aureus (MRSA) bacteria.

Also disclosed herein are articles of manufacture, comprising: a compound as disclosed herein, and a container. Further articles of manufacture include articles of manufacture, comprising: a compound as disclosed herein, a container; and instructions as to how to administer the compound.

In an embodiment, Applicants disclose herein a pyridazinone compound having the structure:

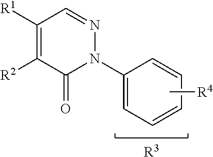

Wherein:

R1 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R2 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R3 is alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and, where R3 is phenyl or cyclohexyl, and

The pyridazinone compound has five R4 substituents, wherein R4 is independently hydrogen, hydroxyl, halogen, nitroxyl, alkyl, alkenyl, alkynyl, acyl, aryl, cycloalkyl, cycloaryl, haloalkyl, alkyloxy, or aryloxy, with the proviso that compounds named herein 2(lead), 2-1, 2-2, 2-5 to 2-10, 2-22, 2-25, 2-27, 2-28, 2-39 and 2-42 to 2-48 are excluded.

In a further embodiment, the pyridazinone compound as disclosed herein has the structure:

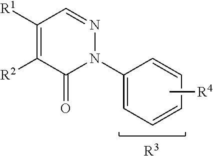

And has substituents wherein:

R1 is halogen, sulfhydryl, sulfoxyl, substituted sulfyl, alkyl-substituted aryl, alkyl substituted cyclohexyl, halogen-substituted aryl, or halogen-substituted cyclohexyl, alkyloxy, or aryloxy;

R2 halogen, sulfhydryl, sulfoxyl, substituted sulfyl, alkyl-substituted aryl, alkyl substituted cyclohexyl, halogen-substituted aryl, or halogen-substituted cyclohexyl, alkyloxy, or aryloxy;

R3 is haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and, where R3 is phenyl or cyclohexyl, and

The pyridazinone compound has five R4 substituents, wherein R4 is independently hydrogen, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy.

In a further embodiment, the pyridazinone compound having the structure

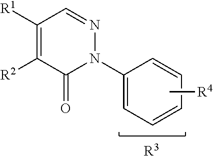

as disclosed herein has substituents wherein:

R1 is halogen, sulfhydryl, sulfoxyl, substituted sulfyl, or alkyloxy;

R2 halogen, sulfhydryl, sulfoxyl, substituted sulfyl, or alkyloxy;

R3 is phenyl or cyclohexyl; and

R4 is hydrogen, halogen, nitroxyl, alkyl, alkenyl, alkynyl, acyl, aryl, haloalkyl, cycloalkyl, cycloaryl, alkyloxy, or aryloxy.

In a still further embodiment, the pyridazinone compound having the structure:

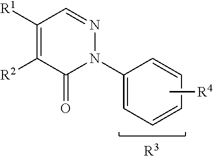

as disclosed herein has substituents wherein:

R1 and R2 are independently halogen, sulfhydryl, sulfoxyl, aryl-substituted sulfhydryl, —S—S—R5, wherein R5 is hydrogen, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R3 is phenyl or cyclohexyl; and

R4 is hydrogen, halogen, nitroxyl, alkyl, alkenyl, alkynyl, acyl, aryl, haloalkyl, cycloalkyl, cycloaryl, alkyloxy, or aryloxy.

In embodiments, Applicants disclose herein a pyridazinone compound having the structure:

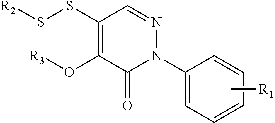

Wherein

Five R1 substituents are independently hydrogen, hydroxyl, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R2 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and

R3 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy.

In an embodiment, Applicants disclose herein a compound selected from the compounds named herein 2-3, 2-11, 2-12, 2-13, 2-14, 2-15, 2-16, 2-17, 2-18, 2-19, 2-20, 2-21, 2-23, 2-24, 2-26, 2-29, 2-30, 2-31, 2-32, 2-33, 2-34, 2-35, 2-36, 2-37, 2-38, 2-40, 2-41, 2-49 and 2-50 (see, e.g., Table 2).

In an embodiment, Applicants disclose herein a pyridazinone compound having the structure selected from:

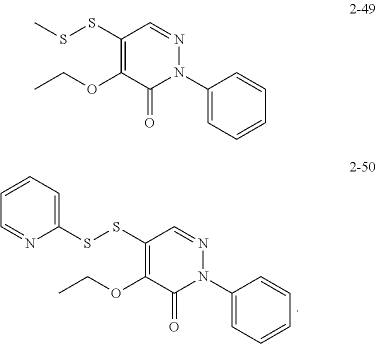

In an embodiment, Applicants disclose herein a pyridazinone compound selected from

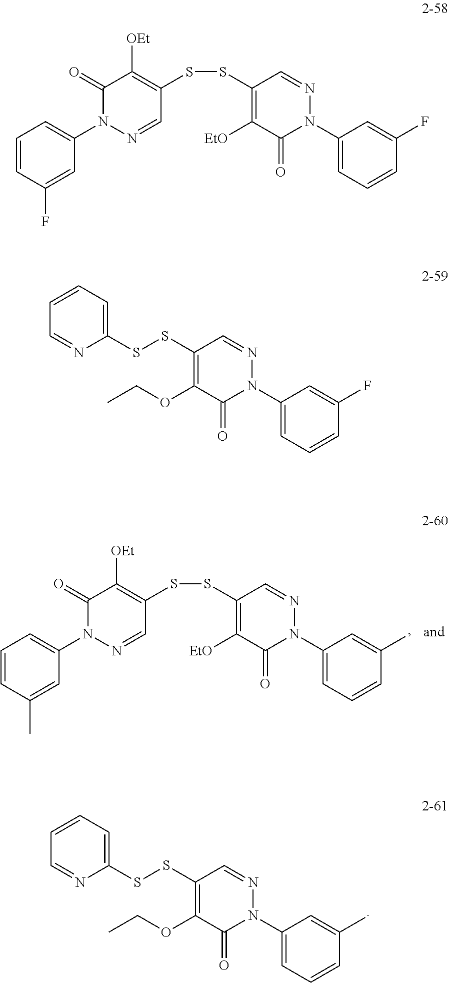

In an embodiment, Applicants disclose herein a rhodanine compound having the structure:

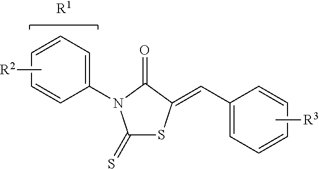

Wherein

R1 is hydrogen, hydroxyl, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R2 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and

R3 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy,

with the proviso that compounds named herein 1(lead), 1-1, 1-2, 1-3, 1-4, 1-5, 1-6, and 1-7 are excluded.

In an embodiment, Applicants disclose herein a rhodanine compound having the structure:

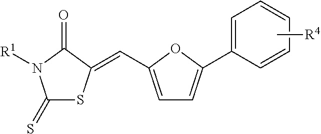

Wherein

R1 is hydrogen, hydroxyl, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and

R4 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy, with the proviso that compounds named herein 1-8, 1-9, 1-10, 1-12, and 1-13 are excluded (see, e.g., Table 1).

In an embodiment, Applicants disclose herein a pyrazolethione compound having the structure:

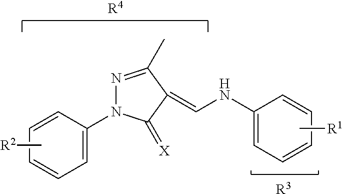

Wherein

X is O or S;

Five R1 substituents are independently hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R2 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;

R3 is cyclohexyl, cycloaryl, substituted cycloaryl, substituted cyclohexyl, pyridinyl, alkyl-substituted aryl, alkyl substituted cyclohexyl, halogen-substituted aryl, or halogen-substituted cyclohexyl; and

R4 includes any suitable R2 and X, with the proviso that compounds named herein 3(lead), 3-1, 3-2, 3-3, 3-4, 3-5, 3-6, 3-7, 3-8, 3-9, 3-10, 3-11, 3-12, 3-13, 3-14, 3-15, 3-16, 3-17, 3-18, 3-19, 3-20, and 3-21 are excluded (see, e.g., Table 3).

In an embodiment, Applicants disclose herein the compound having the structure:

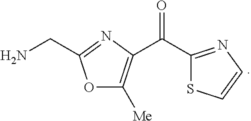

In an embodiment, Applicants disclose herein a compound selected from

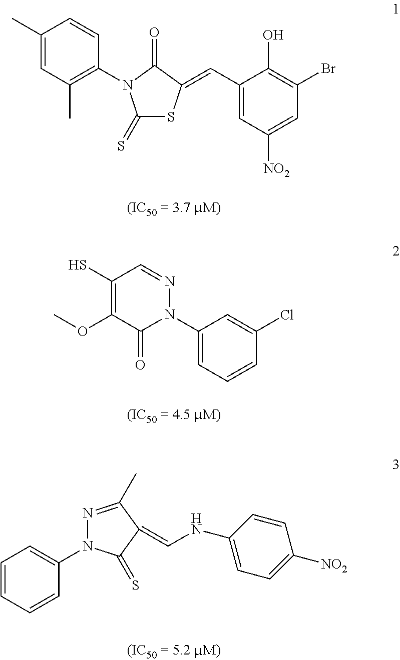

In an embodiment, Applicants disclose herein a pharmaceutical composition comprising an effective amount of a compound as disclosed herein, in admixture with a pharmaceutically acceptable carrier.

In an embodiment, Applicants disclose herein a pharmaceutical composition comprising an effective amount of a pyridazinone compound as disclosed herein, in admixture with a pharmaceutically acceptable carrier.

In an embodiment, Applicants disclose herein the use of the compound as disclosed herein, for treating an infectious disorder.

In an embodiment, Applicants disclose herein the use of the compound as disclosed herein to formulate a medicament for treating an infectious disorder.

In an embodiment, Applicants disclose herein a method of making a pyridazinone compound, comprising steps of:

Adding a thiol solution to an ethanol-containing solution of compound having the structure I, to provide a compound having the structure II,

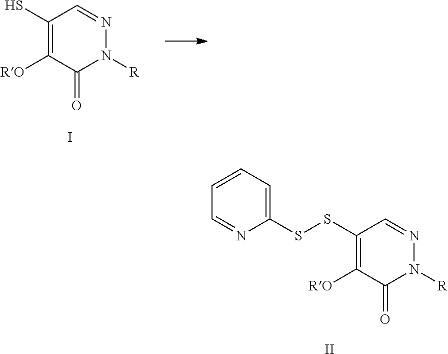

Wherein R is cyclohexyl, phenyl, alky, alkenyl, alkynyl, acyl, acyloxy, aryl, aryloxy, alkyl-substituted aryl, alkyl substituted cyclohexyl, halo, halogen-substituted aryl, or halogen-substituted cyclohexyl; and

Wherein R′ is cyclohexyl, phenyl, alky, alkenyl, alkynyl, acyl, acyloxy, aryl, aryloxy, alkyl-substituted aryl, alkyl substituted cyclohexyl, halo, halogen-substituted aryl, or halogen-substituted cyclohexyl.

In an embodiment, Applicants disclose herein a method of making a pyridazinone compound comprising steps of:

adding a compound having the structure I to an ethanol-containing solution of compound having the structure II, providing a mixture in said ethanol-containing solution having a ratio of approximately 3 parts structure I to 2 parts structure II, to provide a compound having the structure III,

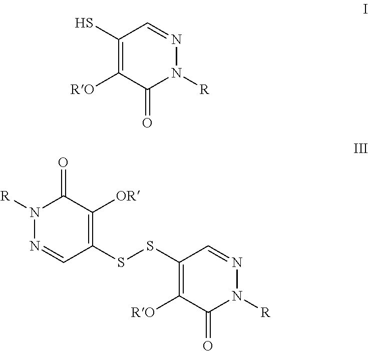

Wherein R is cyclohexyl, phenyl, alky, alkenyl, alkynyl, acyl, acyloxy, aryl, aryloxy, alkyl-substituted aryl, alkyl substituted cyclohexyl, halo, halogen-substituted aryl, or halogen-substituted cyclohexyl; and

Wherein R′ is cyclohexyl, phenyl, alky, alkenyl, alkynyl, acyl, acyloxy, aryl, aryloxy, alkyl-substituted aryl, alkyl substituted cyclohexyl, halo, halogen-substituted aryl, or halogen-substituted cyclohexyl.

In an embodiment, Applicants disclose herein a method of treating a subject in need of treatment, comprising administering an effective dose of a pharmaceutical composition as disclosed herein.

In an embodiment, Applicants disclose herein the method of treating a subject in need of treatment comprises treatment for a bacterial infection. In an embodiment, the method of treating a subject in need of treatment, comprising treatment for a bacterial infection comprises treatment of an infection of a gram positive bacterium. In embodiments, the gram positive bacterium is selected from the group of bacteria consisting of Staphylococcus aureus (S. Aureus; SA), Listeria monocytogenes, Corynebacterium diphtheriae, Enterococcus faecalis, Clostridium perfringen, Clostridium tetani, Streptococcus pyogenes and Streptococcus pneumoniae, Bacillus anthracis (B. anthracis; BA). In embodiments, the gram positive bacterium is a Methicillin resistant Staphylococcus aureus (MRSA) bacterium.

In an embodiment, Applicants disclose herein an article of manufacture, comprising: a compound as disclosed herein, and a container.

In a further embodiment, Applicants disclose herein an article of manufacture, comprising: a compound as disclosed herein, a container, and instructions as to how to administer the compound.

Accordingly, the compounds, compositions, uses, formulations, medicaments, articles of manufacture and methods disclosed herein provide advantages over the art.

### FIGURE LEGENDS

FIG. 1. (A) FRET assay for measuring SrtA enzymatic activity. Three progress curves are overlaid and correspond to inhibitors with different potencies. (B) Histogram showing the distribution of 30,000 compounds in the ChemBridge library as a function of % inhibition of SrtA determined by an end-point analysis during the high-throughput screening campaign. (C) Venn diagram showing how the initial velocity (vᵢ) and end-point analyses were used to identify 44 inhibitors of S. aureus SrtA. Lead compounds 1-3 were selected from these inhibitors and have the best physicochemical and inhibitory properties. The number of compounds in each population is shown in parentheses.

FIG. 2. Structures of the SrtA inhibitors identified by high-throughput screening. The IC₅₀ value against S. aureus SrtA of each compound is indicated.

FIG. 3. Additional asymmetric disulfide derivatives synthesized for the pyridazinone series containing thiomethyl (2-49) or 2-thiopyridyl (2-50) groups. IC₅₀ values against S. aureus SrtA are indicated.

FIG. 4. Inhibition of S. aureus cell growth by the lead compounds and several potent inhibitor compounds identified in the SAR studies. Growth inhibition was measured using the microtiter broth dilution method. In this procedure 180 μL of the cell culture was plated into a 96 well plate and 20 μL of inhibitor solution was added to a final concentration of 500 μM. Growth was then monitored overnight at 37° C. using a temperature-controlled plate reader. The % growth inhibition is relative to cultures grown in the absence of inhibitor. Error bars are the standard deviation from three measurements.

FIG. 5. Image showing the SrtA-inhibitor complexes generated by Induced-Fit Docking. Dock poses with the highest rank (lowest IFD score value) are shown. Compounds 1 (A), 2 (B), 2-1 (C), 2-35 (D), 3 (E), and 3-12 (F) were docked into the structure of S. aureus SrtA derived from the solution structure of the covalent complex between SrtA and the LPAT sorting signal analog (Suree, N.; Liew, C. K.; Villareal, V. A.; Thieu, W.; Fadeev, E. A.; Clemens, J. J.; Jung, M. E.; Clubb, R. T. 2009, (JBC submitted)). Ligand structures are shown in a ‘ball and stick” format. The solvent accessible surface of SrtA is shown and colored to indicate the electrostatic properties from acidic (red) to basic (blue). The secondary structure of the protein is shown behind the surface and the important neighboring amino acids are labeled. The figures were created using the program PyMOL (DeLano, W. L. The PyMOL Molecular Graphics System; 0.99 ed.; DeLano Scientific: South San Francisco).

FIG. 6. Rationally designed inhibitor of sortase A (SrtA) (compound 4). The IC₅₀ of compound 4 for inhibiting SrtA is 7.2 μM.

FIG. 7. Proposed mechanisms of SrtA catalysis for thLPXTG substrate (left) and for the rationally designed inhibitor (right). The label “Enz” indicates a portion of the SrtA enzyme.

FIG. 8. Cell adhesion assay used to measure SrtA activity in whole cells. The figure shows adherence of wild-type and srtA-S. aureus strains to IgG coated microtiter plates. The potent effects of compound 4 (+Cpd4) are shown.

FIG. 9. Effect of increasing concentration of compound 2-50 on cellulase activity are shown. Sortase activity was determined by using cellulase activity.

FIG. 10. Effects of compounds 2-50, 2-59, 3-12, and 3-17 on cellulase activity are shown, to determine effects on sortase activity. The concentration of each compound was twenty-fold greater than the previously determined IC₅₀ value for that compound. At these concentrations, about 30% to about 40% of the sortase activity was inhibited.

FIG. 11 1D-NMR spectra for compound 2-42

FIG. 12 1D-NMR spectra for compound 2-43

FIG. 13 1D-NMR spectrum for compound 2-44

FIG. 14 1D-NMR spectra for compound 2-45

FIG. 15 1D-NMR spectra for compound 2-46

FIG. 16 1D-NMR spectra for compound 2-47

FIG. 17 1D-NMR spectra for compound 2-48

FIG. 18 1D-NMR spectra for compound 2-22

FIG. 19 1D-NMR spectra for compound 2-23

FIG. 20 1D-NMR spectra for compound 2-24

FIG. 21 1D-NMR spectra for compound 2-25

FIG. 22 1D-NMR spectra for compound 2-26

FIG. 23 1D-NMR spectra for compound 2-27

FIG. 24 1D-NMR spectra for compound 2-28

FIG. 25 1D-NMR spectra for compound 2-29

FIG. 26 1D-NMR spectra for compound 2-30

FIG. 27 1D-NMR spectra for compound 2-31

FIG. 28 1D-NMR spectra for compound 2-32

FIG. 29 1D-NMR spectra for compound 2-33

FIG. 30 1D-NMR spectra for compound 2-34

FIG. 31 1D-NMR spectrum for compound 2-35

FIG. 32 1D-NMR spectrum for compound 2-36

FIG. 33 1D-NMR spectra for compound 2-37

FIG. 34 1D-NMR spectra for compound 2-38

FIG. 35 1D-NMR spectra for compound 2-39

FIG. 36 1D-NMR spectrum for compound 2-40

FIG. 37 1D-NMR spectra for compound 2-41

FIG. 38 1D-NMR spectra for compound 2-10

FIG. 39 1D-NMR spectrum for compound 2-11

FIG. 40 1D-NMR spectra for compound 2-12

FIG. 41 1D-NMR spectra for compound 2-13

FIG. 42 1D-NMR spectra for compound 2-14

FIG. 43 1D-NMR spectra for compound 2-15

FIG. 44 1D-NMR spectra for compound 2-16

FIG. 45 1D-NMR spectrum for compound 2-18

FIG. 46 1D-NMR spectrum for compound 2-19

FIG. 47 1D-NMR spectra for compound 2-20

FIG. 48 1D-NMR spectrum for compound 2-21

FIG. 49 1D-NMR spectra for compound 2-17

FIG. 50 1D-NMR spectra for compound 2-49

FIG. 51 1D-NMR spectra for compound 2-50

FIG. 52 NOESY spectrum for compound 2-10

FIG. 53 NOESY spectrum for compound 2-18

Table 1 provides structural and srtA inhibition information regarding exemplary srtA-inhibiting rhodanine compounds. SA indicates S. Aureus; BA indicates B. Anthracis.

Table 2 provides structural and srtA inhibition information regarding exemplary srtA-inhibiting pyridazinone compounds. SA indicates S. Aureus; BA indicates B. Anthracis.

Table 3 provides structural and srtA inhibition information regarding exemplary srtA-inhibiting pyazolethione compounds. SA indicates S. Aureus; BA indicates B. Anthracis.

Table 4 provides structural and srtA inhibition information regarding exemplary srtA-inhibiting pyridazinone compounds 2-58, 2-59, 2-60, and 2-61.

Table 5 provides structural and melting point information for several exemplary compounds.

### DETAILED DESCRIPTION

Described herein are compounds capable of effectively treating bacterial infections by inhibiting the sortase A (SrtA) protein in Staphylococcus aureus and/or related enzymes in other gram positive bacteria, such as the pathogen Bacillus anthracis. In some aspects, compounds provided herein belong to the classes of compounds commonly termed pyridazinones, rhodanines and pyrazolethiones. In some aspects, the rhodanines are exemplified by 1, the pyridazinones are exemplified by 2-9 and the pyrazolethione compounds are exemplified by 3-12.

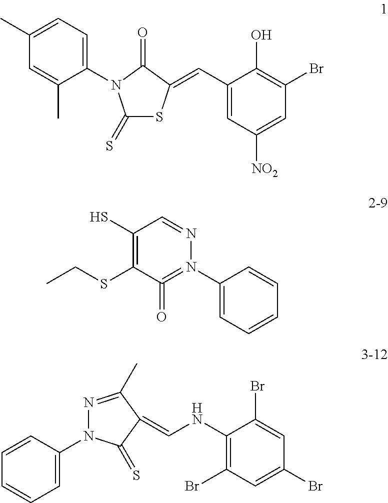

Compounds described herein are potent inhibitors of the SrtA sortase enzymes from S. aureus and B. anthracis. Many of the compounds inhibit the activity of these enzymes with IC₅₀ values in the high nanomolar range and are 10 to 100 times more active than previously reported compounds. For example, compounds 2-9 and 3-12 inhibit the enzyme with IC₅₀ values of 1.4 and 0.3 μM, respectively, and molecules based on the pyridazinone frame work can reach IC₅₀ values of about 0.20 μM. In particular examples, compounds 2-58, 2-59, 2-60, and 2-61 (also based on the pyridazinone frame work):

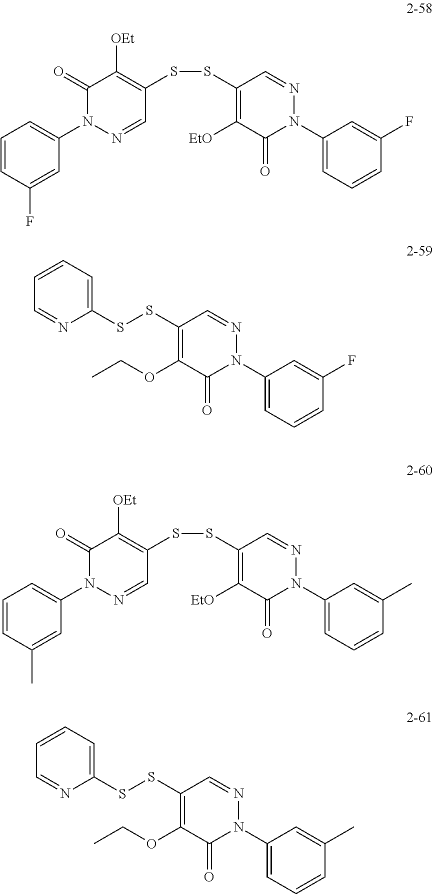

inhibit the enzyme with IC₅₀ values of 0.16, 0.04, 0.14, and 0.07 μM, respectively (see Table 4).

Compounds provided herein are advantageous over currently used antibiotics as they do not impair microbial growth in cell culture, indicating that they are unlikely to generate selective pressures that lead to the evolution of microbes with drug resistance. Moreover, compounds provided herein exhibit minimum inhibitory concentrations (MIC) in the millimolar range. This indicates that the compounds will function as anti-infective agents, preventing microbial growth in the human host, while not hindering growth outside of the human host. Compounds provided herein are useful for treating a range of bacterial infections, especially those caused by Methicillin-resistant Staphylococcus aureus (MRSA).

The compounds disclosed herein find use in inhibiting srtA, in treating gram positive bacterial infections, in preparing pharmaceutical formulations and in manufacturing medicaments for treating gram positive bacterial infections. However, in embodiments, some compounds disclosed herein may not be included in a group, or in groups of compounds which may be selected for inclusion in pharmaceutical formulations for such treatments, or for use in such treatments, or for use in the manufacture of such medicaments. For example, compounds 2-1, 2-2, 2-5 to 2-10, 2-22, 2-25, 2-27, 2-28, 2-39 and 2-42 to 2-48 may, in embodiments of the inventions disclosed herein, be excluded from a group, or from groups, selected for inclusion in pharmaceutical formulations for such treatments, or for use in such treatments, or for use in the manufacture of such medicaments. In a further example, all the 3 compounds, e.g., 3-1, etc., may, in embodiments of the inventions disclosed herein, be excluded from a group, or from groups, selected for inclusion in pharmaceutical formulations for such treatments, or for use in such treatments, or for use in the manufacture of such medicaments. In yet a further example, the first eight rhodanine compounds (e.g., 1-1, 1-2, 1-3 etc. to 1-8), may, in embodiments of the inventions disclosed herein, be excluded from a group, or from groups, selected for inclusion in pharmaceutical formulations for such treatments, or for use in such treatments, or for use in the manufacture of such medicaments.

The descriptions of various embodiments of the invention are presented for purposes of illustration, and are not intended to be exhaustive or to limit the invention to the forms disclosed. Persons skilled in the relevant art can appreciate that many modifications and variations are possible in light of the embodiment teachings.

It should be noted that the language used herein has been principally selected for readability and instructional purposes, and it may not have been selected to delineate or circumscribe the inventive subject matter. Accordingly, the disclosure is intended to be illustrative, but not limiting, of the scope of invention.

It must be noted that, as used in the specification, the singular forms “a,” “an” and “the” include plural referents unless the context clearly dictates otherwise.

As used herein, the term “IC₅₀” has its usual meaning of indicating the concentration at which the inhibition by a test compound is half-maximal.

As used herein, the term “EC₅₀” has its usual meaning of indicating the concentration at which the effect of a test compound is half-maximal.

The compounds disclosed herein are useful in the treatment of infectious disorders comprising infection by gram positive bacteria having sortase A. Such infections include, for example, bacterial infections of the lung, such as, e.g., bacterial pneumonia.

Gram positive bacteria include Staphyloccus, Streptococcus, Enterococcus, Bacillus, Corynebacterium, Nocardia, Clostridium, Actinobacteria, and Listeria bacteria. Sortase A is found in a wide range of bacterial genera, including among others: Bacillus, Enterococcus, Lactobacillus, Lactococcus, Listeria, Staphylococcus, and Streptococcus genera. For example, gram positive bacteria which have sortase A include Staphylococcus aureus (S. Aureus; SA), Listeria monocytogenes, Corynebacterium diphtheriae, Enterococcus faecalis, Clostridium perfringen, Clostridium tetani, Streptococcus pyogenes and Streptococcus pneumoniae, Bacillus anthracis (B. anthracis; BA). Other bacteria which are believed to have sortase A include: Actinomyces naeslundii, Actinomyces viscosus, Arcanobacterium pyogenes, Arthrobacter sp., Bacillus sp., Clostridium septicum, Desulfitobacterium hafniense, Erysipelothrix rhusiopathiae, Lactobacillus leichmannii, Lactobacillus paracasei, Lactobacillus reuteri, Listeria grayi, Listeria seeligeri, Peptostreptococcus magnus (Finegoldia magna), Staphylococcus carnosus, Staphylococcus lugdunensis, Staphylococcus saprophyticus, Staphylococcus warneri, Staphylococcus xylosus, Streptococcus constellatus, Streptococcus criceti, Streptococcus downei, Streptococcus dysgalactiae, Streptococcus intermedius, Streptococcus parasanguinis, Streptococcus salivarius, and Streptococcus thermophilus.

In embodiments, the invention provides for both prophylactic and therapeutic treatment of infectious disorders.

In one embodiment, the invention provides a method of treating a bacterial infection, such as an infectious disorder in a mammal comprising administering to the mammal an effective amount of a compound as disclosed herein.

In another aspect, the invention encompasses the foregoing method of treating bacterial infectious disorder wherein the compound is a pyridazinone compound as disclosed herein. In embodiments, the pyridazinone compound is compound 2-58, 2-59, 2-60, or 2-61, or a compound having a structure closely related to, or derived from, compound 2-58, 2-59, 2-60, or 2-61.

Definitions and Nomenclature

The terminology used herein is for the purpose of describing particular embodiments only and is not intended to be limiting.

It must be noted that, as used in the specification and the appended claims, the singular forms “a,” “an” and “the” include plural referents unless the context clearly dictates otherwise.

As used herein, “pg” means picogram, “ng” means nanogram, “μg” means microgram, “mg” means milligram, “μl” means microliter, “ml” means milliliter, “l” means liter.

“Optional” or “optionally” means that the subsequently described circumstance may or may not occur, so that the description includes instances where the circumstance occurs and instances where if does not.

The terms “active agent,” “drug” and “pharmacologically active agent” are used interchangeably herein to refer to a chemical material or compound which, when administered to an organism (human or animal, generally human) induces a desired pharmacologic effect. In the context of the present invention, the terms generally refer to a hydrophobic therapeutic active agent, preferably fenofibrate, unless the context clearly indicates otherwise.

“Pharmaceutically acceptable” means suitable for use in mammals, i.e., not biologically or otherwise undesirable. Thus, for example, the phrase “pharmaceutically acceptable” refers to molecular entities and compositions that are physiologically tolerable and do not typically produce an allergic or similar untoward reaction, such as gastric upset, dizziness and the like, when administered to a human.

A “salt” refers to all salt forms of a compound, including salts suitable for use in industrial processes, such as the preparation of the compound, and pharmaceutically acceptable salts.

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid. Examples of pharmaceutically acceptable salts are described in Berge, S. M. et al., “Pharmaceutical Salts,” Journal of Pharmaceutical Science, 1977; 66:1 19.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” means an alleviation of symptoms associated with an infection, halt of further progression or worsening of those symptoms, or prevention or prophylaxis of the infection. Treatment can also include administering the compounds and pharmaceutical formulations of the present invention in combination with other therapies. For example, the compounds and pharmaceutical formulations of the present invention can be administered before, during, or after surgical procedure and/or radiation therapy. The compounds of the invention can also be administered in conjunction with other antibacterial drugs, or with other drugs and treatments that may, or may not, be directed to the treatment of bacterial infections.

“Subject” or “patient” as used herein refers to a mammalian, preferably human, individual who can benefit from the pharmaceutical compositions and dosage forms of the present invention.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight and general condition of the subject, the severity of the condition being treated, the judgment of the clinician, and the like. Thus, it is not possible to specify an exact “effective amount.” However, an appropriate “effective amount” in any individual case may be determined by one of ordinary skill in the art using only routine experimentation.

The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier.

Any embodiment described herein can be combined with any other suitable embodiment described herein to provide additional embodiments. For example, where one embodiment individually or collectively describes possible groups for R₁, R₂, R₃, R₄, R₅, etc., and a separate embodiment describes possible R₇ groups, it is understood that these embodiments can be combined to provide an embodiment describing possible groups for R₁, R₂, R₃, R₄, R₅, etc. with the possible R₇ groups, etc. With respect to the above compounds, and throughout the application and claims, the following terms have the meanings defined below.

“Substituted” refers to a group in which one or more bonds to a hydrogen atom contained therein are replaced by a bond to non-hydrogen atom. In some instances the bond will also be replaced by non-carbon atoms such as, but not limited to: a halogen atom such as F, Cl, Br, and I; a nitrogen atom in groups such as amines, amides, alkylamines, dialkylamines, arylamines, alkylarylamines, diarylamines, heterocyclylamine, (alkyl)(heterocyclyl)amine, (aryl)(heterocyclyl)amine, or diheterocyclylamine groups, isonitrile, N-oxides, imides, and enamines; an oxygen atom in groups such as hydroxyl groups, alkoxy groups, aryloxy groups, ester groups, and heterocyclyloxy groups; a silicon atom in groups such as in trialkylsilyl groups, dialkylarylsilyl groups, alkyldiarylsilyl groups, and triarylsilyl groups; a sulfur atom in groups such as thiol groups, alkyl and aryl sulfide groups, sulfone groups, sulfonyl groups, and sulfoxide groups; and other heteroatoms in various other groups. Substituted alkyl groups and substituted cycloalkyl groups also include groups in which one or more bonds to one or more carbon or hydrogen atoms are replaced by a bond to a heteroatom such as oxygen in carbonyl, carboxyl, and ether groups; nitrogen in groups such as imines, oximes and hydrazones. Substituted cycloalkyl, substituted aryl, substituted heterocyclyl and substituted heteroaryl also include rings and fused ring systems which can be substituted with alkyl groups as described herein. Substituted arylalkyl groups can be substituted on the aryl group, on the alkyl group, or on both the aryl and alkyl groups. All groups included herein, such as alkyl, alkenyl, alkylene, alkynyl, aryl, heterocyclyl, heterocyclyloxy, and the like, can be substituted. Representative examples of substituents for substitution include one or more, for example one, two or three, groups independently selected from halogen, —OH, —C₁₋₆ alkyl, C₁₋₆ alkoxy, trifluoromethoxy, —S(O)ₙC₁₋₆ alkyl, amino, haloalkyl, thiol, cyano, —OR₁0 and —NR₈R₉, and trifluoromethyl.

The phrase “acyl” refers to groups having a carbon double-bonded to an oxygen atom, such as in the structure —C(═O)R. Examples of R can include H, such as in aldehydes, a hydrocarbon, such as in a ketone, —NR₈R₉, such as in an amide, —OR₆ such as in a carboxylic acid or ester, —OOCR₂, such as in an acyl anhydride or a halo, such as in an acyl halide.

The phrase “alkenyl” refers to straight and branched chain hydrocarbons, such as those described with respect to alkyl groups described herein, that include at least one double bond existing between two carbon atoms. Examples include vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others. An alkenyl group can optionally be substituted, for example where 1, 2, 3, 4, 5, 6, 7, 8 or more hydrogen atoms are replaced by a substituent selected from the group consisting of halogen, haloalkyl, hydroxy, thiol, cyano, and —NR₈R₉.

The phrase “alkyl” refers to hydrocarbon chains, for example C₁₋₆ chains, that do not contain heteroatoms. Thus, the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂ CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃), —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The phrase includes primary alkyl groups, secondary alkyl groups, and tertiary alkyl groups. Alkyl groups can be bonded to one or more carbon atom(s), oxygen atom(s), nitrogen atom(s), and/or sulfur atom(s) in the parent compound. An alkyl group can optionally be substituted, for example where 1, 2, 3, 4, 5, 6 or more hydrogen atoms are replaced by a substituent selected from the group consisting of halogen, haloalkyl, hydroxy, thiol, cyano, and —NR₈R₉.

The phrase “alkylene” refers to a straight or branched chain divalent hydrocarbon radical, generally having from two to ten carbon atoms.

The phrase “alkynyl” refers to straight and branched chain hydrocarbon groups, such as those described with respect to alkyl groups as described herein, except that at least one triple bond exists between two carbon atoms. Examples include —C≡C(H), —C≡C(CH₃), —C≡C(CH₂CH₃), —C(H₂)C≡C(H), —C(H)₂C≡C(CH₃), and —C(H)₂C≡C(CH₂CH₃) among others. An alkynyl group can optionally be substituted, for example where 1, 2, 3, 4, 5, 6, 7, 8 or more hydrogen atoms are replaced by a substituent selected from the group consisting of halogen, haloalkyl, hydroxy, thiol, cyano, and —NR₈R₉.

The phrase “aminoalkyl” refers to an alkyl group as above attached to an amino group, which can ultimately be a primary, secondary or tertiary amino group. An example of an amino alkyl group is the —NR₈R₉ where one or both of R₈ and R₉ is a substituted or unsubstituted C₁₋₆ alkyl or R₈ and R₉ together with the atom to which they are attached form a substituted or unsubstituted heterocyclic ring. Specific aminoalkyl groups include —NHCH₃, —N(CH₃)₂, —NHCH₂CH₃, —N(CH₃)CH₂CH₃, —N(CH₂CH₃)₂, —NHCH₂CH₂CH₃, —N(CH₂CH₂CH₃)₂, and the like.

An aminoalkyl group can optionally be substituted with 1, 2, 3, 4 or more non-hydrogen substituents, for example where each substituent is independently selected from the group consisting of halogen, cyano, hydroxy, C₁₋₆ alkyl, C₁₋₆ alkoxy, C₁₋₂ alkyl substituted with one or more halogens, C₁₋₂ alkoxy substituted with one or more halogens, —C(O)R₆, —C(O)OR₆, —S(O)ₙR₆ and —NR₈R₉. These substituents may be the same or different and may be located at any position of the ring that is chemically permissible.

The phrase “aryl” refers to cyclic or polycyclic aromatic rings, generally having from 5 to 12 carbon atoms. Thus the phrase includes, but is not limited to, groups such as phenyl, biphenyl, anthracenyl, naphthenyl by way of example. The phrase “unsubstituted aryl” includes groups containing condensed rings such as naphthalene. Unsubstituted aryl groups can be bonded to one or more carbon atom(s), oxygen atom(s), nitrogen atom(s), and/or sulfur atom(s) in the parent compound. Substituted aryl groups include methoxyphenyl groups, such as para-methoxyphenyl.

Substituted aryl groups include aryl groups in which one or more aromatic carbons of the aryl group is bonded to a substituted and/or unsubstituted alkyl, alkenyl, alkynyl group or a heteroatom containing group as described herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, and hydroxyphenyl among others. An aryl moiety can optionally be substituted with 1, 2, 3, 4 or more non-hydrogen substituents, for example where each substituent is independently selected from the group consisting of halogen, cyano, hydroxy, C₁₋₆ alkyl, C₁₋₆ alkoxy, C₁₋₂ alkyl substituted with one or more halogens, C₁₋₂ alkoxy substituted with one or more halogens, —C(O)R₆, —C(O)OR₆, —S(O)ₙR₆ and —NR₈R₉. These substituents may be the same or different and may be located at any position of the ring that is chemically permissible.

The phrase “cycloalkyl” refers to cyclic hydrocarbon chains, generally having from 3 to 12 carbon atoms, and includes cyclic alkyl groups such as cyclopropyl, cyclobutyl, cyclopentyl, cyclohexyl, cycloheptyl, and cyclooctyl and such rings substituted with straight and branched chain alkyl groups as described herein. The phrase also includes polycyclic alkyl groups such as, but not limited to, adamantanyl, norbornyl, and bicyclo[2.2.2]octyl and such rings substituted with straight and branched chain alkyl groups as described herein. Cycloalkyl groups can be saturated or unsaturated and can be bonded to one or more carbon atom(s), oxygen atom(s), nitrogen atom(s), and/or sulfur atom(s) in the parent compound. A cycloalkyl group can be optionally substituted, for example where 1, 2, 3, 4 or more hydrogen atoms are replaced by a substituent selected from the group consisting of halogen, cyano, hydroxy, C₁₋₆ alkyl, C₁₋₆ alkoxy, C₁₋₂ alkyl substituted with one or more halogens, C₁₋₂ alkoxy substituted with one or more halogens, —C(O)R₆, —C(O)OR₆, —S(O)ₙR₆ and —NR₈R₉.

The term “Ph” refers to phenyl.

The phrase “halo” refers to a halide, e.g., fluorine, chlorine, bromine or iodine.

The phrase “haloalkyl” refers to an alkyl group in which at least one, for example 1, 2, 3, 4, 5 or more, hydrogen atom(s) is/are replaced with a halogen. Examples of suitable haloalkyls include chloromethyl, difluoromethyl, trifluoromethyl, 1-fluro-2-chloro-ethyl, 5-fluoro-hexyl, 3-difluro-isopropyl, 3-chloro-isobutyl, etc.

The phrases “heterocyclyl” or “heterocyclic ring” refers to aromatic, nonaromatic, saturated and unsaturated ring compounds including monocyclic, bicyclic, and polycyclic ring compounds, including fused, bridged, or spiro systems, such as, but not limited to, quinuclidyl, containing 1, 2, 3 or more ring members of which one or more is a heteroatom such as, but not limited to, N, O, P and S. Unsubstituted heterocyclyl groups include condensed heterocyclic rings such as benzimidazolyl. Examples of heterocyclyl groups include: unsaturated 3 to 8 membered rings containing 1 to 4 nitrogen atoms such as, but not limited to pyrrolyl, pyrrolinyl, imidazolyl, imidazolidinyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; saturated 3 to 8 membered rings containing 1 to 3 oxygen atoms such as, but not limited to, tetrahydrofuran; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as; but not limited to, dihydrooxathiinyl; saturated 3 to 8 membered rings containing 1 to 2 oxygen atoms, and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl groups also include those described herein in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Heterocyclyl groups can contain 5 or 6 ring members. Examples of heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

A heterocyclyl group can be optionally substituted, for example where 1, 2, 3, 4 or more hydrogen atoms are replaced by a substituent selected from the group consisting of halogen, cyano, hydroxy, C₁₋₆ alkyl, C₁₋₆ alkoxy, C₁₋₂ alkyl substituted with one or more halogens, C₁₋₂ alkoxy substituted with one or more halogens, —C(O)R₆, —C(O)OR₆, —S(O)ₙR₆ and —NR₈R₉. Examples of “substituted heterocyclyl” rings include 2-methylbenzimidazolyl, 5-methylbenzimidazolyl, 5-chlorobenzthiazolyl, 1-methylpiperazinyl, and 2-chloropyridyl among others. Any nitrogen atom within a heterocyclic ring can optionally be substituted with C₁₋₆ alkyl, if chemically permissible.

Heterocyclyl groups include heteroaryl groups as a subgroup. The phrase “heteroaryl” refers to a monovalent aromatic ring radical, generally having 5 to 10 ring atoms, containing 1, 2, 3, or more heteroatoms independently selected from S, O, or N. The term heteroaryl also includes bicyclic groups in which the heteroaryl ring is fused to a benzene ring, heterocyclic ring, a cycloalkyl ring, or another heteroaryl ring. Examples of heteroaryl include 7-benzimidazolyl, benzo[b]thienyl, benzofuryl, benzothiazolyl, benzothiophenyl, 2-, 4-, 5-, 6-, or 7-benzoxazolyl, furanyl, furyl, imidazolyl, indolyl, indazolyl, isoquinolinyl, isothiazolyl, isoxazolyl, oxadiazolyl, oxazolyl, purinyl, pyrazinyl, pyrazolyl, pyridazinyl, pyridinyl, pyrimidinyl, pyrrolyl, quinolinyl, tetrazolyl, thiadiazolyl, thiazolyl, thienyl, thiophenyl, triazolyl and the like. Heteroaryl rings can also be optionally fused to one or more of another heterocyclic ring(s), heteroaryl ring(s), aryl ring(s), cycloalkenyl ring(s), or cycloalkyl rings. A heteroaryl group can be optionally substituted, for example where 1, 2, 3, 4 or more hydrogen atoms are replaced by a substituent selected from the group consisting of halogen, cyano, hydroxy, C₁₋₆ alkyl, C₁₋₆ alkoxy, C₁₋₂ alkyl substituted with one or more halogens, C₁₋₂ alkoxy substituted with one or more halogens, —C(O)R₆, —C(O)OR₆, —S(O)ₙR₆ and —NR₈R₉.

The phrase “heterocyclyloxy” refers to a group in which an oxygen atom is bound to a ring atom of a heterocyclyl group as described herein.

The term “protected” with respect to hydroxyl groups, amine groups, and sulfhydryl groups refers to forms of these functionalities which are protected from undesirable reaction with a protecting group known to those skilled in the art such as those set forth in Protective Groups in Organic Synthesis, Greene, T. W.; Wuts, P. G. M., John Wiley & Sons, New York, N.Y., (3rd Edition, 1999) which can be added or removed using the procedures set forth therein. Examples of protected hydroxyl groups include silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include amides such as, formamide, acetamide, trifluoroacetamide, and benzamide; imides, such as phthalimide, and dithiosuccinimide; and others. Examples of protected sulfhydryl groups include thioethers such as S-benzyl thioether, and S-4-picolyl thioether; substituted S-methyl derivatives such as hemithio, dithio and aminothio acetals; and others.

Although not always necessary, the compositions of the present invention may also include one or more additional components, i.e., carriers or additives (as used herein, these terms are interchangeable). When present, however, such additional components may act as an adjuvant to facilitate the formation and maintenance of a pharmaceutically acceptable composition. Classes of additives that may be present in the compositions, include, but are not limited to, absorbents, acids, adjuvants, anticaking agent, glidants, antitacking agents, antifoamers, anticoagulants, antimicrobials, antioxidants, antiphlogistics, astringents, antiseptics, bases, binders, chelating agents, sequestrants, coagulants, coating agents, colorants, dyes, pigments, compatibilizers, complexing agents, softeners, crystal growth regulators, denaturants, dessicants, drying agents, dehydrating agents, diluents, dispersants, emollients, emulsifiers, encapsulants, enzymes, fillers, extenders, flavor masking agents, flavorants, fragrances, gelling agents, hardeners, stiffening agents, humectants, lubricants, moisturizers, bufferants, pH control agents, plasticizers, soothing agents, demulcents, retarding agents, spreading agents, stabilizers, suspending agents, sweeteners, disintegrants, thickening agents, consistency regulators, surfactants, opacifiers, polymers, preservatives, antigellants, rheology control agents, UV absorbers, tonicifiers and viscomodulators. One or more additives from any particular class, as well as one or more different classes of additives, may be present in the compositions. Specific examples of additives are well known in the art.

The pharmaceutical compositions of the present invention are prepared by conventional methods well known to those skilled in the art. The composition can be prepared by mixing the active agent with an optional additive according to methods well known in the art. Excess solvent or solubilizer, added to facilitate solubilization of the active agent and/or mixing of the formulation components, can be removed before administration of the pharmaceutical dosage form. The compositions can be further processed according to conventional processes known to those skilled in the art, such as lyophilization, encapsulation, compression, melting, extrusion, balling, drying, chilling, molding, spraying, spray congealing, coating, comminution, mixing, homogenization, sonication, cryopelletization, spheronization and granulation to produce the desired dosage form.

Therapeutic formulations of the compounds and compositions may be prepared for storage by mixing the compound having the desired degree of purity with optional physiologically acceptable carriers, excipients, or stabilizers, in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as ethylene diamine tetra acetic acid (EDTA); sugar alcohols such as mannitol or sorbitol; salt-forming counterions such as sodium; and/or nonionic surfactants such as Tween, Pluronics or polyethylene glycol (PEG).

The pharmaceutical composition may be prepared as a single dosage form. The dosage form(s) are not limited with respect to size, shape or general configuration, and may comprise, for example, a capsule, a tablet or a caplet, or a plurality of granules, beads, powders or pellets that may or may not be encapsulated. In addition, the dosage form may be a drink or beverage solution or a spray solution that is administered orally. Thus, for example, the drink or beverage solution may be formed by adding a therapeutically effective amount of the composition in, for example, a powder or liquid form, to a suitable beverage, e.g., water or juice.

For example, a dosage form may be a capsule containing a composition as described herein. The capsule material may be either hard or soft and is generally made of a suitable compound such as gelatin, starch or a cellulosic material. As is known in the art, use of soft gelatin capsules places a number of limitations on the compositions that can be encapsulated. See, for example, Ebert (1978), “Soft Elastic Gelatin Capsules: A Unique Dosage Form,” Pharmaceutical Technology 1(5). Two-piece hard gelatin capsules are preferably sealed, such as with gelatin bands or the like. See, for example, Remington: The Science and Practice of Pharmacy, Nineteenth Edition. (1995), or later editions of the same, which describes materials and methods for preparing encapsulated pharmaceuticals. In this embodiment, the encapsulated composition may be liquid or semi-solid (e.g., a gel).

For dosage forms substantially free of water, i.e., when the composition is provided in a pre-concentrated form for administration or for later dispersion in an aqueous system, the composition is prepared by simple mixing of the components to form a pre-concentrate. Compositions in liquid or semi-solid form can be filled into soft gelatin capsules using appropriate filling machines. Alternatively, the composition can also be sprayed, s granulated or coated onto a substrate to become a powder, granule or bead that can be further encapsulated or tableted if the compositions solidify at room temperature with or without the addition of appropriate solidifying or binding agents.

The compound to be used for in vivo administration must be sterile. This is readily accomplished by filtration through sterile filtration membranes, e.g., prior to or following lyophilization and reconstitution. Compositions comprising a compound having features of the invention generally are placed into a container having a sterile access port, for example, an intravenous solution bag or vial having a stopper pierceable by a hypodermic injection needle. The compound may be stored in lyophilized form or in solution. The compound may be stored in a suitable aqueous or solvent solution.

In accordance with the present invention, the pharmaceutical compositions and dosage forms can be administered to treat patients. Patients suffering from any condition, disease or disorder which can be effectively treated with sortase A inhibitors can benefit from the administration of a therapeutically effective amount of the sortase A inhibitor-containing compositions described herein. In particular, however, the sortase A inhibitor-containing compositions are effective in treating bacterial infections, particularly gram positive bacterial infections, such as Staphylococcus aureus infections.

A wide range of bacterial infections may be treated by sortase A inhibitors, as indicated by studies that have shown that genetically modified pathogens that are unable to produce sortase are less virulent or otherwise deficient in processes presumed to be important for pathogenesis. Thus, in addition to diseases caused by S. aureus, diseases that may be treated with sortase A inhibitors include, for example, Streptococcal Diseases (Streptococcus pyogenes), which includes mild diseases such as strep throat or skin infections (impetigo), as well as severe illnesses such as necrotizing faciitis, streptococcal toxic shock syndrome and rheumatic fever. Further diseases that may be treated with sortase A inhibitors include, for example, Streptococcal diseases (S. agalactiae), including such diseases as pneumonia and meningitis in neonates and in the elderly, and systemic bacteremia. Further diseases that may be treated with sortase A inhibitors include, for example, S. pneumoniae, a leading cause of bacterial pneumonia and occasional etiology of otitis media, sinusitis, meningitis and peritonitis. Yet further diseases that may be treated with sortase A inhibitors include, for example, Bacillus anthracis, the causative agent of anthrax. Still further diseases that may be treated with sortase A inhibitors include, for example, life-threatening nosocomial infections caused by E. faecalis. Further diseases that may be treated with sortase A inhibitors include, for example, infections caused by the food-borne pathogen Listeria monocytogenes.

Administration of compounds and compositions as disclosed herein may be via topical, oral (including sublingual), inhalational, intraocular, or other route; may be by injection or infusion (e.g., intravenous, intra-arterial, intramuscular, intraperitoneal, intracerebroventricular, epidermal, or other route of injection), by enema or suppository (e.g., rectal or vaginal suppository), by sustained release system, or by any other means or combination of means of administration as is known in the art.

The composition may be administered in the form of a capsule wherein a patient swallows the entire capsule. Alternatively, the composition may be contained in capsule which is then opened and mixed with an appropriate amount of aqueous fluid such as water or juice to form a drink or beverage for administration of the composition. As will be appreciated, the composition need not be contained in a capsule and may be housed in any suitable container, e.g., packets, ampules, etc. Once prepared, the drink or beverage is imbibed in its entirety thus effecting administration of the composition. Preparation of the composition-containing drink or beverage may be effected by the patient or by another, e.g., a caregiver. As will be appreciated by those skilled in the art, additional modes of administration are available.

Compositions may be prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

For the prevention or treatment of disease, the appropriate dosage of a pharmaceutical composition comprising a sortase A inhibitor compound as disclosed herein, will depend on the pharmaceutical composition employed, the type of disease to be treated, the severity and course of the disease, whether the pharmaceutical composition is administered for preventive or therapeutic purposes, previous therapy, the patient's clinical history and response to the pharmaceutical composition, and the discretion of the attending physician. Typically the clinician will administer the pharmaceutical composition until a dosage is reached that achieves the desired result.

Suitable dosages will be in a range commensurate with the IC₅₀ of the particular compound, where an effective dose provides a plasma concentration, in a subject to which the compound has been administered, that is at least equal to, or preferably greater than, the IC₅₀ of the particular compound for inhibiting sortase A. In embodiments, a dosage will be in a range effective to provide a plasma concentration in a subject to which the compound has been administered of between about 0.01 micromolar (μM) and about 100 μM, or between about 0.02 μM and about 50 μM, or between about 0.03 μM and about 30 μM, or between about 0.05 μM and about 10 μM.

For example, the pharmaceutical composition is suitably administered to the patient at one time or over a series of treatments. Depending on the type and severity of the disease, a dosage effective to provide about 0.01 micromolar (μM) and about 100 μM, or between about 0.05 μM and about 10 μM of the compound in the plasma of a patient is an initial candidate dosage for administration to the patient, whether, for example, by one or more separate administrations, or by continuous or repeated dosing. A typical daily dosage might range from about 0.1 μg/kg to 100 mg/kg or more, depending on the factors mentioned above. For example, in embodiments a typical daily dosage might range from about 0.1 mg/kg to about 1 mg/kg. For repeated administrations over several days or longer, depending on the condition, the treatment is sustained until a desired suppression of disease symptoms occurs. A preferred dosing regimen comprises administering an initial dose of about 1 μg/kg to about 10 mg/kg, or in embodiments from about 0.1 mg/kg to about 1 mg/kg, followed by a weekly maintenance dose of about 0.1 μg/kg to about 1 mg/kg, or in embodiments, from about 0.1 mg/kg to about 1 mg/kg, of the pharmaceutical composition. However, other dosage regimens may be useful, depending on the pattern of pharmacokinetic decay that the practitioner wishes to achieve. The progress of this therapy is easily monitored by conventional techniques and assays.

It is to be understood that while the invention has been described in conjunction with the preferred specific embodiments thereof, that the description above as well as the examples which follow are intended to illustrate and not limit the scope of the invention. Other aspects, advantages and modifications within the scope of the invention will be apparent to those skilled in the art to which the invention pertains.

The practice of the present invention will employ, unless otherwise indicated, conventional techniques of pharmaceutical formulation, medicinal chemistry, and the like, which are within the skill of the art. Such techniques are explained fully in the literature. Preparation of various types of pharmaceutical formulations are described, for example, in Remington: The Science and Practice of Pharmacy, Nineteenth Edition. (1995) and Ansel et al., Pharmaceutical Dosage Forms and Drug Delivery Systems, 6.sup.th Ed. (Media, Pa.: Williams & Wilkins, 1995).

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric. All components were obtained commercially unless otherwise indicated.

All patents, patent applications, and publications mentioned herein, both supra and infra, are hereby incorporated by reference.

Any terms not directly defined herein shall be understood to have the meanings commonly associated with them as understood within the art of the invention. Certain terms are discussed herein to provide additional guidance to the practitioner in describing the compositions, devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the embodiments of the invention herein.

High-Throughput Screening Identifies Several SrtA Inhibitors.

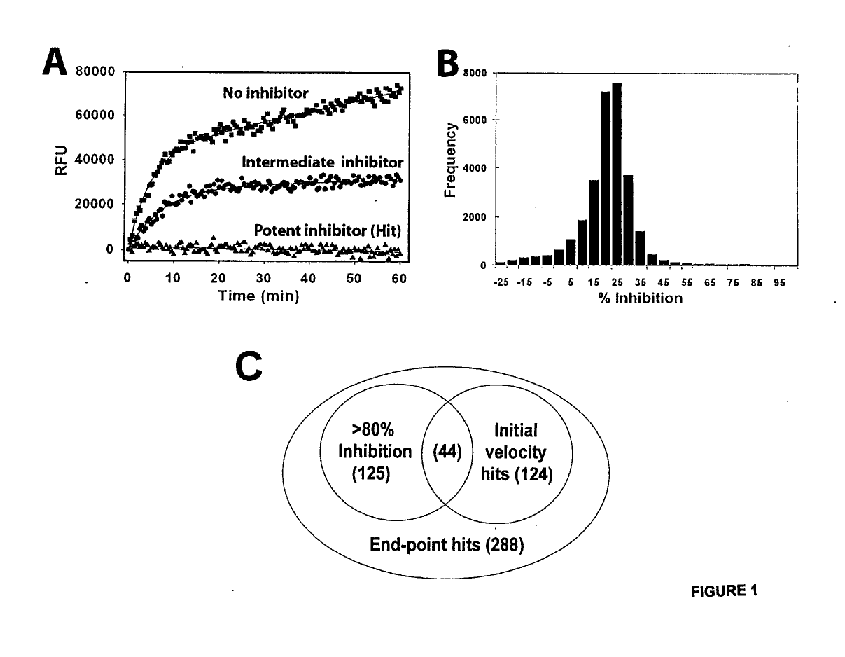

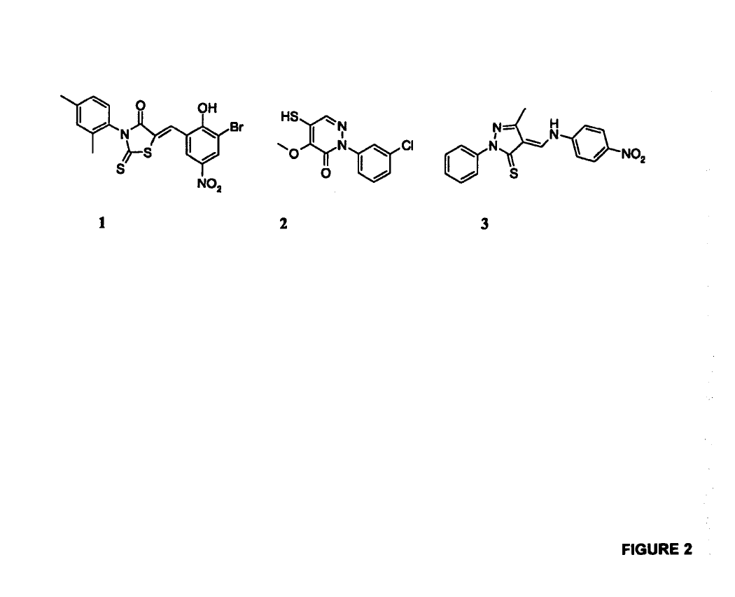

In order to screen for small molecule inhibitors of SrtA we modified a fluorescence resonance energy transfer (FRET) assay that monitors the SrtA-catalyzed hydrolysis of an internally quenched fluorescent substrate analogue (o-aminobenzoyl (Abz)-LPETG-diaminopropionic acid-dinitrophenyl-NH₂ (Dap(Dnp)). The assay was miniaturized to enable its use in high-throughput screening (HTS). A typical progress curve is shown in FIG. 1A. The calculated Z′ score (a statistical measure of the assay's robustness) is 0.75, which indicates that the assay can be effectively used for screening (Zhang, J. H.; Chung, T. D.; Oldenburg, K. R. J. Biomol. Screen. 1999, 4, 67). The DiverSet library (ChemBridge Corp.) was screened for inhibitors of SrtA (see experimental section). Two criteria were used to calculate the inhibition percentage (% inhibition) of each compound in the library: (1) the initial velocity (vᵢ) of product formation calculated from reaction progress curves, and (2) an end-point determination of product formation obtained by measuring the total product fluorescence five hours after initiating the reaction. Compounds in the library were first ranked by their end-point readings. This revealed a Gaussian distribution (FIG. 1B), such that molecules that exhibit >55% enzyme inhibition can be considered as hits with a 99.7% confidence limit (their % inhibition value is at least three standard deviation units above the mean) (Copeland, A. R. Evaluation of Enzyme Inhibitors in Drug Discoveries; John Wiley & Sons: New Jersey, 2005). A total of 288 compounds met this criterion. The number of potential inhibitors was then further reduced by selecting only those molecules for which >80% inhibition was observed in the end-point analysis, as well as statistically significant inhibition when their vᵢ values were considered (the vᵢ value was less than or equal to 0 based on a 10 minute progress curve). This reduced the total number of compounds to 44 (FIG. 1C). Their inhibitory activity was then confirmed by manually repeating the FRET assay and they were ranked based on their % inhibition as determined by the end-point analysis. From this set, ten compounds were selected for further study because they had the highest inhibitory activity and because they had physicochemical properties similar to known drugs (Lajiness, M. S.; Vieth, M.; Erickson, J. Curr. Opin. Drug Discov. Devel. 2004, 7, 470; Viswanadhan, V. N.; Balan, C.; Hulme, C.; Cheetham, J. C.; Sun, Y. Curr. Opin. Drug Discov. Devel. 2002, 5, 400; Darvas, F.; Keseru, G.; Papp, A.; Dorman, G.; Urge, L.; Krajcsi, P. Curr. Top. Med. Chem. 2002, 2, 1287; Lipinski, C. A.; Lombardo, F.; Dominy, B. W.; Feeney, P. J. Adv. Drug Deliv. Rev. 2001, 46, 3; Lipinski, C. A.; Hoffer, E. Compound properties and drug quality. Practice of Medicinal Chemistry; 2 ed., 2003; 341; Lipinski, C. A. Drug Discov. Today: Technol. 2004, 1, 337). For these inhibitors, the concentration that is required to reduce the activity of SrtA by 50% (IC₅₀) was determined using well established methods (Kim, S. W.; Chang, I. M.; Oh, K. B. Biosci. Biotechnol. Biochem. 2002, 66, 2751; Copeland, A. R. Evaluation of Enzyme Inhibitors in Drug Discoveries; John Wiley & Sons: New Jersey, 2005; Huang, X.; Aulabaugh, A.; Ding, W.; Kapoor, B.; Alksne, L.; Tabei, K.; Ellestad, G. Biochemistry 2003, 42, 11307). The most potent SrtA inhibitors from this group are shown in FIG. 2 (compounds 1-3) and were chosen for further study.

Analysis of the Reversibility of Inhibition of SrtA.

For the three lead molecules, the reversibility of enzyme inhibition was determined by measuring the enzymatic activity of each enzyme-inhibitor complex immediately after it was rapidly diluted (Copeland, A. R. Evaluation of Enzyme Inhibitors in Drug Discoveries; John Wiley & Sons: New Jersey, 2005). In this study SrtA was first incubated with saturating concentrations of each compound (inhibitor concentrations 10-fold higher than the IC₅₀ value). The SrtA-inhibitor complexes were then rapidly diluted and the enzyme activity immediately measured (data not shown). Inhibition by compound 1 is rapidly reversible as 84% of the enzyme activity is recovered after dilution. Compounds 2 and 3 also reversibly inhibit the enzyme, but more slowly; 50% and 58% activity is regained immediately after dilution, respectively. Mass spectrometry was also employed to confirm that the molecules form a reversible complex with the enzyme (described in the Experimental section). In this study, the mass spectrum of each saturated SrtA-inhibitor complex was recorded 1, 48, or 96 hours after forming the complex. Mass spectra of these enzyme-inhibitor complexes showed no difference from the negative control (SrtA alone), suggesting that the inhibitors do not stably modify the enzyme. In addition, detailed studies on inhibitory reversibility of the lead compounds and their derivatives have also been conducted.

Structure Activity Relationship (SAR) Analysis

An SAR analysis of the three lead compounds (1, 2, and 3, see FIG. 2) was performed to identify related molecules with increased potency. Initially, we purchased closely-related analogs of the lead compounds from the ChemBridge Corp. and determined their IC₅₀ values against S. aureus SrtA. The analogs were identified through search of the company's database and share 75-95% similarity (based on the chemical functionality and scaffolding as determined by the company's similarity search engine) with one of the three lead compounds. A total of 7, 9 and 21 analogs of lead compounds 1, 2 and 3 were purchased and tested, respectively. This work enabled regions of the chemical scaffold required for inhibition to be coarsely defined. Analogs of the rhodanine 1 and pyridazinone 2 were then synthesized to make more subtle changes to discover molecules with even higher potency or better physicochemical properties. Eight analogs of 1 ((compounds 1-8 to 1-13)) and a total of 41 analogs of 2 were produced and tested (compounds 2-10 to 2-50). Tables 1-3 show the structures of all of the compounds that were tested and their IC₅₀ numbers. To gain insights into their selectivity, for several of the compounds we also measured their IC₅₀ values against the Bacillus anthracis sortase enzyme (^{Ba}SrtA). A discussion of this data is presented below.

Synthesis and SAR of the Rhodanine Compounds (Series 1)

Two scaffolds of the rhodanine compounds were examined by SAR (Table 1). Compounds with scaffold A were purchased from ChemBridge Corp. (1 to 1-8), while compounds with scaffold B were synthesized in our laboratory (1-8 to 1-13). The synthesis of these compounds followed literature precedence, namely reaction of the N-alkyl isothiocyanate with methyl thioglycolate gave the 3-alkyl-4-oxothiazolidine-2-thiones. Condensation of these with the 5-arylfurfuraldehydes gave the compounds 1-8 to 1-13 in good yields (Condon, F. E.; Shapiro, D.; Sulewski, P.; Vasi, I.; Waldman, R. Org. Prep. Proc. Int. 1974, 6, 37-43; Drobnica, L.; Knoppova, V.; Komanova, E. Chem. Zvesti 1972, 26, 538-42). In scaffold A, replacing the 2,4-dimethyl groups on the R² position reduces the potency 3-5 fold (cpd. 1 vs. 1-1, 1-2, 1-3, 1-7). On the other hand, relocating the 2-OH group on the R³ position reduces the potency by 10-fold (cpd. 1 vs 1-4). These data suggest that these functional groups play a critical role in enzyme binding, presumably through hydrophobic interaction via the 2,4-dimethyl groups on the R² position, and hydrogen bonding via the 2-OH group at the R³ position. The SAR results for compounds with scaffold B are in general agreement with this interpretation. Although these molecules retain the central rhodanine nucleus, they differ in the R¹ group and replace the R³ group with a much larger 5-phenyl furan moiety. Similar to the results obtained for the scaffold A molecules, these variations result in molecules with significantly elevated IC₅₀ values. The most dramatic difference can be seen by comparing compounds 1 and 1-10. Even though they are closely related on one side of the rhodanine ring (Ph vs CH₂Ph on the R¹ position), the other side is substantially different as compound 1-10 does not have the aforementioned 2-OH group. Taken together, none of the analogs of compound 1 showed improved activity against SrtA and were not pursued further.

Synthesis and SAR of the Pyridazinone Compounds (Series 2)

Initial SAR studies of lead compound 2 made use of derivatives purchased from ChemBridge (compounds 2-1 to 2-9) (Table 2). This work revealed one of the most potent inhibitors of SrtA, compound 2-1 (Kᵢ^{app}=0.20 where Kᵢ^{app} is the apparent dissociation constant for the enzyme-inhibitor complex, as determined by the Morrison's equation) (Copeland, A. R. Evaluation of Enzyme Inhibitors in Drug Discoveries; John Wiley & Sons: New Jersey, 2005) and its close analog 2-9 (Kᵢ^{app}=1.4 μM). This discovery led us to investigate variants of these compounds by synthesizing several analogs (2-10 to 2-50). These compounds were prepared by an adaptation of the literature route, (Liga, J. W. J. Heterocyc. Chem. 1988, 25, 1757-1760) namely heating a mixture of an arylhydrazine, mucochloric acid, and dilute HCl afforded the 2-aryl-4,5-dichloropyridazin-3-ones 2-42 to 2-48 in good yields (85-95%). The less reactive 4-nitrophenylhydrazine required more forcing conditions, namely a toluene solution of the initial formed hydrazone cyclization toluene was heated at reflux for 10 h using a Dean-Stark to afford the analogue 2-43 in 76% yield for the two steps. The regioselectivity of the addition of oxygen nucleophiles to 2-42 to 2-48 was dependent on the conditions: use of 1,4-dioxane as the solvent, with sodium ethoxide or methoxide, afforded cleanly the 4-alkoxy products 2-22 to 2-34 (83-95% yield) while the use of sodium hydroxide in ethanol afforded cleanly the 5-ethoxy analogues 2-35 to 2-41 (75-94% yield). The assignment of the regiochemistry of the products was based on the observation of a strong NOE enhancement of the methylene of the ethyl signal in the 5-ethoxy compounds with the C5 vinyl hydrogen, an NOE which was absent from the 4-alkoxy compounds. The displacement of the remaining chloride atom in either the 4- or 5-alkoxy compounds was uneventful although we found that the reaction worked best in DMF as solvent. In this way the analogues 2-10 to 2-16 and 2-18 to 2-21 were formed. The symmetrical disulfide dimer, 2-17, could be formed by direct air oxidation of the thiol 2-10. The other disulfides were prepared by the reaction of the thiol 2-10 with methyl methanethiosulfonate (MMTS) or Aldrichthiol (2-pyridyldisulfide) to give 2-49 and 2-50 in yields of 88% and 65%, respectively. Finally the symmetrical disulfide 2-17 could also be prepared in 85% yield by reaction of the thiol 2-10 with the pyridyl disulfide 2-50.

Substituents on the pyridazinone ring (R¹ and R²) were suspected to contribute greatly to the inhibitory activity, as replacing the —SH with —OH at the R¹ position dramatically reduces potency (2 vs. 2-7). Minor alteration of R² (from —OMe to —OEt) and removal of 3-Cl on the phenyl ring (R⁴) also increase the potency more than 20-fold (compare 2 with 2-1). These observations suggest that the functional groups located on the pyridazinone ring may be as critical as those located on the phenyl ring. Therefore, we synthesized analogs with different substituents on the pyridazinone ring to optimize their potency further. Based on the substituent, these compounds are segregated into 4 subclasses: ethoxy-thiol (2-10 to 2-21); methoxy-chloro (2-22 to 2-27); ethoxy-chloro (2-28 to 2-41); and dichloro (2-42 to 2-48) pyridazinone compounds. Additionally, we also varied the R³ and R⁴ positions of each subclass in order to probe the importance of the phenyl ring. With the exception of compound 2-35, members of the ethoxy-thiol subclass are the most potent molecules. Within this series, switching the relative positioning of the R¹ and R² groups does not dramatically affect activity (compare 2-10 with 2-18, or 2-13 with 2-19, or 2-14 with 2-20). In contrast, varying the phenyl ring causes substantial changes in potency, with the lowest IC₅₀ obtained when all substituents are eliminated or when only small substituents are present. Interestingly, replacing entire phenyl ring with a cyclohexyl group did not profoundly alter activity (2-10 vs. 2-16). This suggests that this portion of the ethoxy-thiol molecules may form non-specific hydrophobic interactions with the enzyme, which can be disrupted with groups larger than a phenyl or cyclohexyl ring are present.

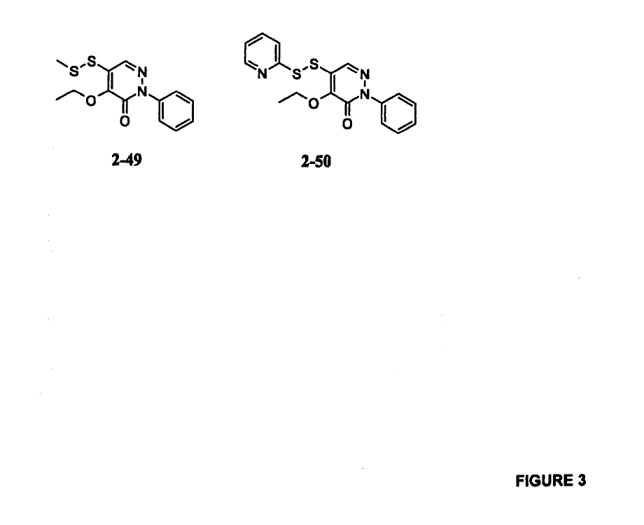

Because the ethoxy-thiol compounds all contain a thiol group that could potentially interact with the active site cysteine thiol of SrtA (residue Cys184) we created a series of molecules that are disulfide variants (compounds 2-17 in table 2, and 2-49, 2-50 in FIG. 3). Compound 2-17 is the symmetrical disulfide dimer of 2-10 and exhibits a about 2-fold increase in its potency. Interestingly, asymmetrical disulfide derivatives of 2-10 that contain methyl (2-49) or pyridyl (2-50) groups are even more potent and exhibit Kᵢ^{app} values of about 0.4 and 0.03 μM, respectively. In this series the pyridyl thiol is the best potential leaving group as it can be transformed into a stabilized pyridine-2-thione. As this derivative is the most potent inhibitor, this data suggest that these molecules may inhibit the enzyme through a thiol-disulfide exchange reaction involving Cys184. However, the mechanism of inhibition by these molecules remains unclear as compound 2 reversibly inhibits SrtA and does not modify the enzyme based on mass spectrometry data (described above). Although the ethoxy-thiol subclass contains several potent SrtA inhibitors, 2-35 within the ethoxy-chloro subclass is nearly as potent with an IC₅₀ value of about 1 μM. This molecule possesses a unique combination of substituents on the pyridazinone ring as it has —OEt and —Cl groups on the R¹ and R² position, respectively. Interestingly, the SAR inhibitory trend observed in the ethoxy-chloro and ethoxy-thiol subclasses differ markedly as variations at the R¹ and R² sites in the ethoxy-chloro subclass result in large reductions in potency that are not observed when similar modifications are made in the ethoxy-thiol subclass. This suggests that compound 2-35 may have a different inhibitory mechanism from the ethoxy-thiol subclass. The binding mode of each molecule was explored further using docking calculations and is discussed later in the text.

SAR of the Pyrazolethione Compounds (Series 3)

A series of pyrazolethione analogues of the lead compound 3 were obtained from ChemBridge through a similarity search. Inhibitory activities against SrtA were evaluated and are shown in Table 3. Initially, substituents on the R¹ ring were varied while we kept the thione group on the pyrazole nucleus constant (compounds 3 to 3-12). This led to the discovery of the most potent compound in the 3-series, 3-12 (Kᵢ^{app}=0.3 μM). This molecule contains a bulky and lipophilic tribromophenyl substituent. Replacing the thione group with a ketone is detrimental (compare 3 with 3-13), while changing substituents on the R² phenyl ring does not significantly restore potency (3-13 vs. 3-14, 3-15, 3-16). We also examined the effect of varying the phenyl ring attached via the amide (R³ and R⁴). These results are obvious; replacement of the phenyl group (R³) with a more electron-withdrawing pyridyl group enhances the potency (compare 3 with 3-17), while a normal cyclohexyl group dramatically reduces the potency (3-18). Variation of the R⁴ group moderately influences inhibitory activity (3-19 to 3-21) with the reduction in potency by a factor of 3-10 compared to the lead, suggesting inhibition may prefer the pyrazolethione nucleus and the phenyl ring on the nitrogen.

The pyrazolethione and pyridazinone compounds also inhibit ^{Ba}SrtA and minimally affect S. aureus growth

In cell culture, srtA⁻ strains of S. aureus show no defects in their growth. This suggests that highly selective SrtA inhibitors will function as anti-infective agents that only prevent the bacterium from thriving within the human host, but otherwise do not impair growth outside of the host. SrtA inhibitors may therefore have advantages over conventional antibiotics that generate selective pressures that lead to their obsolescence. Using a microtiter broth dilution method (Frankel, B. A.; Bentley, M.; Kruger, R. G.; McCafferty, D. G. J. Am. Chem. Soc. 2004, 126, 3404) for lead compounds 1 to 3, we determined the minimal inhibitory concentration (MIC) of each molecule that prevented S. aureus growth. This work revealed that lead compounds 2 and 3 only minimally impair bacterial growth as they have MIC values>1 mM. In contrast, the rhodanine lead compound 1 has an MIC value of about 10 μM, suggesting that it inactivates other reactions essential for bacterial viability. This finding is compatible with recent studies that have shown that rhodanine compounds inhibit class C β-lactamases in Gram-negative bacteria (Grant, E. B.; Guiadeen, D.; Baum, E. Z.; Foleno, B. D.; Jin, H.; Montenegro, D. A.; Nelson, E. A.; Bush, K.; Hlasta, D. J. Bioorg. Med. Chem. Lett. 2000, 10, 2179). Several arylalkylidene rhodanines have also been reported that have high bactericidal activity against non-resistant S. aureus and MRSA strains. These compounds exhibit MIC values lower than ampicillin and cefotaxime and it has been proposed that they noncompetitively inhibit penicillin-binding proteins (Zervosen, A.; Lu, W. P.; Chen, Z.; White, R. E.; Demuth, T. P., Jr.; Frere, J. M. Antimicrob. Agents Chemother. 2004, 48, 961).

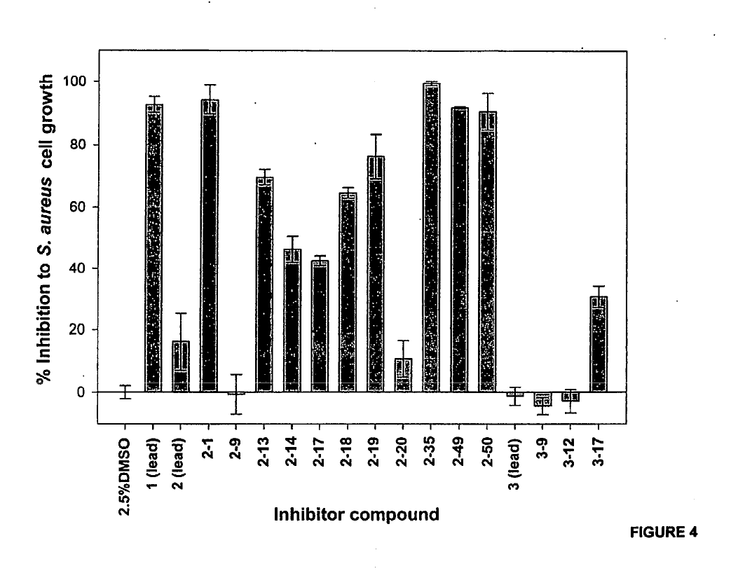

The finding that compounds 2 and 3 do not affect bacterial growth is fortuitous, as nearly all of the potent SrtA inhibitors we identified in the SAR analysis are analogs of these molecules. In order to more rapidly ascertain SrtA inhibitory effects on microbial growth, we grew S. aureus cultures in the presence of 500 μM of each inhibitor and compared the rate of growth with control cultures grown in 2.5% DMSO (the solvent used to solubilize the inhibitors). This method enables an estimate of MIC to be obtained as molecules that do not affect bacterial growth can be assumed to have MIC values>1 mM. Consistent with the MIC data, compound 1 is toxic, while compounds 2 and 3 only modestly perturb growth (FIG. 4). An analysis of the growth data suggests that series 3 molecules are very promising anti-infective agents as four of its molecules inhibit SrtA with an IC₅₀ or Kᵢ^{app}<5 μM, but otherwise do not substantially affect bacterial growth (compounds 3-1, 3-9, 3-12 and 3-17). Interestingly, the most potent SrtA inhibitor (compound 3-12) shows no detrimental effect to bacterial viability, highlighting its potential for further development as an anti-infective agent. Compounds in the 2-series show a variation of effects on S. aureus growth. The most promising candidates for further development are 2-9 and 2-20 as they inhibit SrtA with low micromolar IC₅₀ values and do not significantly inhibit S. aureus growth in cell culture.

The ability of several of the compounds to inhibit the sortase A protein from Bacillus anthracis (^{Ba}SrtA) was tested to gain insights in their selectivity. This enzyme shares 27% amino acid sequence identity with S. aureus SrtA and also attaches proteins to the cell wall that contain an LPXTG sorting signal (Gaspar, A. H.; Marraffini, L. A.; Glass, E. M.; Debord, K. L.; Ton-That, H.; Schneewind, O. J. Bacteriol. 2005, 187, 4646). In addition, ^{Ba}srtA⁻ knockout strains show defects in their ability to escape macrophages, suggesting that ^{Ba}SrtA may be useful in treating anthrax (Zink, S. D.; Burns, D. L. Infect. Immun. 2005, 73, 5222). IC₅₀ measurements against ^{Ba}SrtA were made for the most potent S. aureus SrtA inhibitors. For the series-2 molecules, the S. aureus SrtA and ^{Ba}SrtA enzymes show similar trends in their susceptibility. For example, molecules that poorly inhibit S. aureus SrtA also are ineffective against ^{Ba}SrtA (compounds 2-6 to 2-8), while potent S. aureus SrtA inhibitors also effectively inhibit ^{Ba}SrtA. Interestingly, compounds 2-9 and 2-20, which significantly impair S. aureus SrtA activity and are not bactericidal (FIG. 4), are even more potent ^{Ba}SrtA inhibitors with Kᵢ^{app} values of about 0.3 and 0.4 μM, respectively. The most potent non-bacteriocidal 3-series compounds, 3-9 and 3-12, are also promising, as they inhibit ^{Ba}SrtA with Kᵢ^{app} values of 1.4 and 1.7 μM, respectively. Combined this data suggests that the mechanism of enzyme inhibition by compounds 2-9, 2-20, 3-9 and 3-12 is conserved across species, and that they are unlikely to significantly alter microbial processes other than surface protein display.

Biostructural Analysis

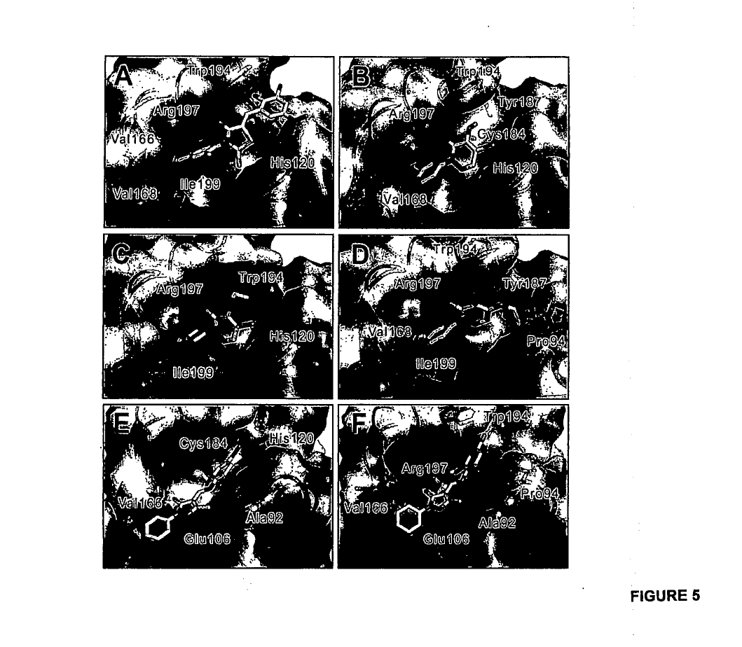

To gain insight into the mode of binding of the SrtA inhibitors, we modeled how they interacted with the S. aureus SrtA enzyme using an Induced-Fit Docking (IFD) protocol (Schrödinger Inc.) (Sherman, W.; Day, T.; Jacobson, M. P.; Friesner, R. A.; Farid, R. J. Med. Chem. 2006, 49, 534; Sherman, W.; Beard, H. S.; Farid, R. Chem. Biol. Drug Des. 2006, 67, 83; Schrödinger Suite 2008; Schrödinger, LLC: New York, N.Y., USA.). Compounds were docked into the recently determined solution structure of SrtA bound to a LPAT peptide (Suree, N.; Liew, C. K.; Villareal, V. A.; Thieu, W.; Fadeev, E. A.; Clemens, J. J.; Jung, M. E.; Clubb, R. T. 2009, (JBC submitted)). After removal of the peptide coordinates the remaining protein structure was prepared for docking using the Protein Preparation Wizard, and LigPrep was used to prepare the ligand compounds (Schrödinger Suite 2008; Schrödinger, LLC: New York, N.Y., USA). The inhibitors were then docked into the SrtA receptor using a standard IFD workflow. Models of the SrtA-inhibitor complexes with the lowest negative IFD value were chosen to represent the final docking solution. When docked into the active site of SrtA, compound 1 inserts its hydrophobic moiety into the lipophilic pocket generated by the side chains of Ile199 in strand β8 and residues Val166 to Val168 in the adjacent β6/β7 loop (FIG. 5A). This may explain why altering the 2,4-Me₂ groups at the R² position reduces potency 3-5 fold. On the rhodanine nucleus, the carbonyl oxygen is positioned toward the highly conserved side chain of Arg197, and its sulfide group is positioned toward His120. On the benzylidene ring, its 2-OH group is in close proximity to Trp194 and Tyr187 side chains, and its 5-NO₂ group is oriented toward His120, suggesting a potential hydrogen bonding network. This could explain the observed dramatic reductions in inhibitory activity when functional groups on the benzylidene ring are relocated (Table 1, alterations to R³).

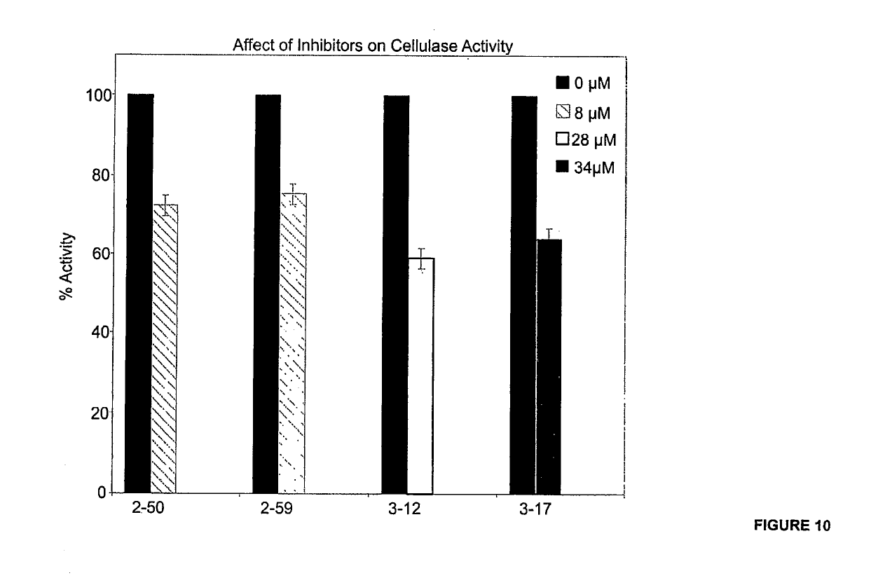

For pyridazinone compounds (series 2), most of them bind to the active site in a similar orientation such that the phenyl ring is buried in the aforementioned lipophilic pocket. This is evident by comparing the docking solutions of compounds 2 (FIG. 5B), 2-1 (FIG. 5C) and 2-35 (FIG. 5D). These models provide a plausible explanation for why compound 2-1 has a Kᵢ^{app} value about 40 fold lower than compound 2, since the chloro group on the ring of compound 2 would seem to create a steric hindrance within this lipophilic pocket. Analogous to the docking solution observed for compound 1 (FIG. 5A), the carbonyl oxygen atom on the pyridazinone ring in the docked complexes of 2, 2-1 and 2-35 are all positioned towards the conserved Arg197 side chain. In addition, the thiol group on both compounds 2 and 2-1 points towards His120, which may explain the significant reduction in activity when this group is replaced with a chloro group (compare ethoxy-thiol with ethoxy-chloro subclasses in table 2). Interestingly, the docking solution of compound 2-35 suggests that it positions its ethoxy moiety toward another lipophilic region created by the side chains of Pro94 and Ala92 located in helix H1. This structural difference may explain the distinct SAR profiles observed within the ethoxy-chloro and ethoxy-thiol subclasses. The ethoxy-thiol subclass is more tolerant to alteration at this site, compatible with the docked solution that projects this group towards an open groove on the protein surface. In contrast, in the ethoxy-chloro series its juxtaposition against the helix H1 may make it less tolerant to alteration, which is compatible with our finding that only compound 2-35 within the ethoxy-chloro series has a low IC₅₀ value (vide supra).

The docking calculations suggest that the elongated structure of the series-3 compounds may be advantageous as it may enable contacts to two hydrophobic pockets on the enzyme. One phenyl ring (R²) is in contact with the β6/β7 loop Val166-Val168 residues, while the other (R³) is closer to Trp194 and Pro94 side chains (FIG. 5E). Changing substituents on this R³ position from 4-NO₂ to 2,4,6-Br₃ (compound 3-12) improved the potency about 15 fold, indicating a preference for a more lipophilic moiety at this position. However, replacing the substituent with 2,4-Me₂ or 3,4-Me₂ reduced potency, suggesting shape complementarity may be critical for binding. The docking solutions also suggest why the pyrazole nucleus may be specific to the sortase active site as its methyl and thione groups contact two highly conserved residues, Ala92 and Arg197, respectively (FIG. 5F). This feature, along with their hydrophobic network, may be the reason why most of the compounds within this series exhibit high potency against SrtA enzymes, but little or no bactericidal activity.

Discussion

Applicants have identified several promising small molecules that reversibly inhibit the S. aureus SrtA sortase with Kᵢ^{app} values in the high nanomolar range, rhodanine, pyrazolethione and pyridazinone compounds. SAR analysis has led to some of the most promising anti-infective agents thus far reported as compounds 2-9 and 3-12 inhibit the enzyme with Kᵢ^{app} values of 1.4 and 0.3 μM, respectively. Importantly, both of these molecules do not impair microbial growth in cell culture, suggesting that they selectively inhibit sortase. Molecules based on the pyridazinone framework are quite promising, and can reach Kᵢ^{app} values of about 0.20 μM, but in some cases were bactericidal. Intriguingly, the most potent inhibitors for S. aureus SrtA also inhibit ^{Ba}SrtA, suggesting further that they are specific sortase inhibitors. Additional studies with more distantly related enzymes will be needed to define the degree of specificity.

The library screening also revealed several rhodanine related compounds that are potent SrtA inhibitors, although some analogs of the lead molecule did not show improved potency. The lead rhodanine compound was also shown to be bactericidal, suggesting it has polytrophic effects. This is consistent with recent studies showing rhodanine compounds inhibit class C β-lactamases in Gram-negative bacteria (Grant, E. B.; Guiadeen, D.; Baum, E. Z.; Foleno, B. D.; Jin, H.; Montenegro, D. A.; Nelson, E. A.; Bush, K.; Hlasta, D. J. Bioorg. Med. Chem. Lett. 2000, 10, 2179) and penicillin-binding proteins in non-resistant S. aureus and MRSA strains (Zervosen, A.; Lu, W. P.; Chen, Z.; White, R. E.; Demuth, T. P., Jr.; Frere, J. M. Antimicrob. Agents Chemother. 2004, 48, 961).

Overall, the biostructural analysis of the inhibitors is in reasonable agreement with the SAR results, and provides insights into the mode of action of each inhibitor from the docking poses. This agreement may in part be due to the use of the recently reported NMR structure of SrtA bound to a (2R,3S) 3-amino-4-mercapto-2-butanol analog of the sorting signal (Suree, N.; Liew, C. K.; Villareal, V. A.; Thieu, W.; Fadeev, E. A.; Clemens, J. J.; Jung, M. E.; Clubb, R. T. 2009, (JBC submitted). The structure of the active site in this protein differs markedly from previously reported structures of the apo-form of the enzyme (PDB:1t2p) (Zong, Y.; Bice, T. W.; Ton-That, H.; Schneewind, O.; Narayana, S. V. J. Biol. Chem. 2004, 279, 31383) and may be more biological relevant. This assertion is substantiated by trial docking experiments using the apo-form of the enzyme that failed to yield results consistent with the SAR data. The structure of the enzyme in its substrate bound form may therefore be useful for virtual screening experiments. In summary, we have discovered potent S. aureus and B. anthracia SrtA sortase inhibitors that could be useful anti-infective agents.

### EXAMPLE 1

### Chemistry

Materials were obtained from commercial suppliers and were used without purification. All the moisture sensitive reactions were conducted under argon atmosphere using oven-dried glassware and standard syringe/septa techniques. Most of reactions were monitored with a silica gel TLC plate under UV light followed by visualization with a p-anisaldehyde or ninhydrin staining solution. Some reactions were monitored by a crude ¹H NMR spectrum. ¹H NMR spectra were measured at 400 MHz in CDCl₃ unless stated otherwise and data were reported as follows in ppm (δ) from the internal standard (TMS, 0.0 ppm): chemical shift (multiplicity, integration, coupling constant in Hz.). 2D-NMR experiments (NOESY, COSY and TOCSY) at 500 MHz were performed to confirm the regioselectivity of the substitution reactions. Melting Points of solid compounds were observed on a Thomas Hoover capillary melting point apparatus. Infrared (IR) spectra were recorded on a Nicolet AVATAR 370 spectrometer using liquid films (neat) on NaCl plates. The purity of the new compounds was assessed by several methods: high-field proton and carbon NMR (lack of significant impurities), R_{f} values on TLC (lack of obvious impurities), melting point, and mass spectrometry.

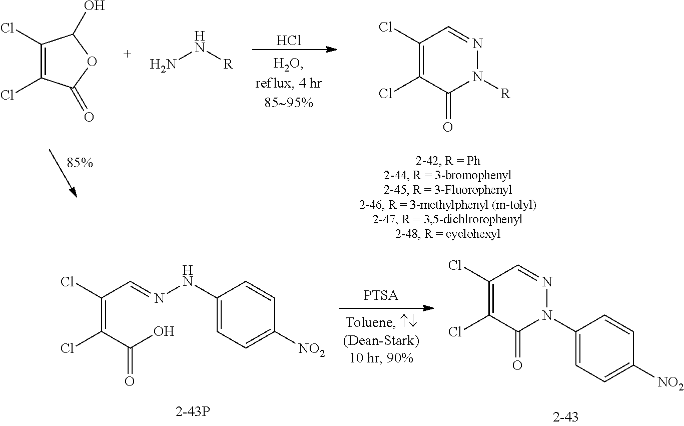

### EXAMPLE 2

### General Procedure for the Synthesis of 2-substituted-4,5-dichloropyridazin-3-ones, e.g., 2-Phenyl-4,5-dichloropyridazin-3-one, 2-42

To a solution of phenyl-hydrazine (2.9 mL, 30 mmol) in diluted HCl (4 M, 60 mL) was added mucochloric acid (5 g, 30 mmol) at 25° C. The solution was refluxed for 3 h. The suspension was filtered and washed with water. The solids were dried under high vacuum to give 7 g of the yellowish white solid, 2-42, 94%. mp 158° C. ¹H NMR δ7.91 (1H, s), 7.57 (2H, m), 7.48 (2H, m), 7.42 (1H, m); ¹³C NMR δ156.15, 140.86, 136.39, 136.14, 135.33, 128.95, 128.89, 125.17.

### EXAMPLE 3

### 2-(4-Nitrophenyl)-4,5-dichloropyridazin-3-one, 2-43

To a solution of 4-nitrophenyl-hydrazine (4.6 mL, 30 mmol) in diluted HCl (4 M, 60 mL) was added mucochloric acid (5 g, 30 mmol) at 25° C. The solution was refluxed for 3 h. The suspension was filtered and washed with water to give the crude 2-43P. The yellow solids were subjected to the following cyclization reaction without further purification. The suspension of the crude 2-43P and p-toluenesulfonic acid (500 mg) in 200 mL of toluene was refluxed for 10 h. The solution was concentrated and the solids were washed with water to give 6.5 g of a yellowish solid, 2-43, 76% (2 steps). mp 221° C. ¹H NMR δ8.35 (2H, d, J=9.2 Hz), 7.98 (1H, s), 7.90 (2H, d, J=9.2 Hz); ¹³C NMR δ155.77, 146.99, 145.37, 136.99, 136.72, 135.65, 125.64, 124.16.

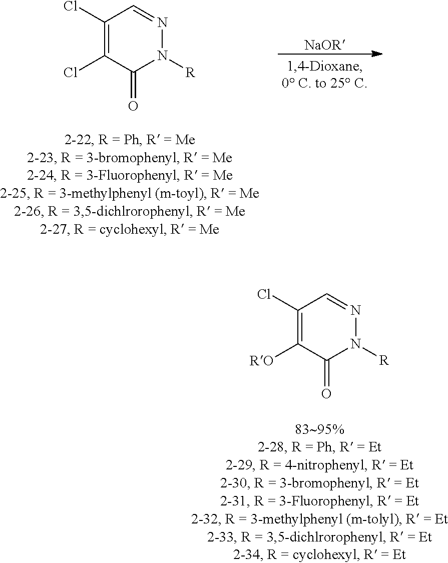

### EXAMPLE 4

### General Procedure for the Synthesis of 2-Substituted 4-Alkoxy-5-chloropyridazin-3-ones, e. g., 5-Chloro-4-ethoxy-2-phenylpyridazin-3-one, 2-28

To a solution of 2-42 (200 mg, 0.809 mmol) in 6 mL of 1,4-dioxane was added 1 mL of freshly generated NaOEt (0.8 M) in EtOH (for methoxy substitution, NaOMe solution in MeOH was used) at 0° C. The suspension was stirred for 2 h as the solution was slowly warmed to 25° C. The suspension was concentrated and the mixture was subjected to flash column chromatography on silica gel to give 189 mg of 2-28, 92%. mp 78° C. ¹H NMR δ7.84 (1H, s), 7.54 (2H, m), 7.48 (2H, m), 7.41 (1H, m); ¹³C NMR δ163.88, 156.01, 140.09, 140.96, 138.17, 128.89, 128.56, 125.46, 123.62, 69.34, 15.94. For the other analogues, the yields varied from 83-95%.

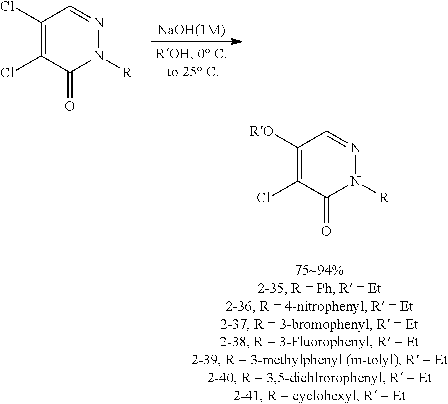

### EXAMPLE 5

### General Procedure for the Synthesis of 2-Substituted 5-Alkoxy-4-chloropyridazin-3-ones, e.g., 4-Chloro-5-ethoxy-2-phenylpyridazin-3-one, 2-35

To a solution of 2-42 (200 mg, 0.809 mmol) in 6 mL of EtOH was added 0.8 mL of NaOH (1 M) at 0° C. The suspension was stirred for 2 h as it was allowed to warm to 25° C. The suspension was concentrated and the mixture was subjected to flash column chromatography on silica gel to give 195 mg of 2-35, 95%. mp 110° C. ¹H NMR δ7.91 (1H, s), 7.57 (2H, m), 7.47 (2H, m), 7.40 (1H, m), 4.38 (2H, q, J=7.2 Hz), 1.54 (3H, t, J=7.2 Hz); ¹³C NMR δ 154.13, 141.22, 132.68, 128.66, 128.32, 127.74, 125.24, 117.34, 66.64, 14.81. For the other analogues, the yields varied from 75-94%.

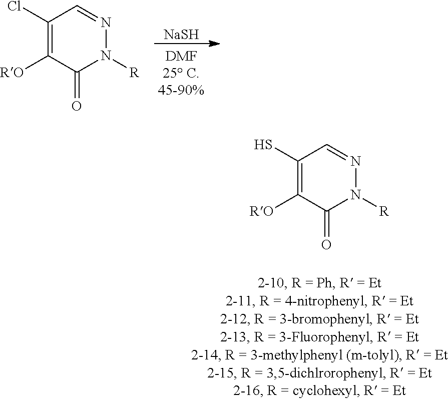

### EXAMPLE 6

### General Procedure for the Synthesis of 2-Substituted 4-Alkoxy-5-mercapto-pyridazin-3-ones, e.g., 4-Ethoxy-5-mercapto-2-phenylpyridazin-3-one, 2-10

To a solution of 2-28 (63 mg, 0.25 mmol) in 2 mL of DMF was added 70 mg of NaSH at 25° C. After TLC showed complete consumption of starting material, the solution was concentrated under high vacuum and diluted with 10 mL of water. The aqueous layer was washed with ethyl acetate and then pH of the aqueous layer was adjusted to 5 about 6 by addition of 1 M HCl (aq). Ethyl acetate (20 mL, two 10 mL portions) was added to the aqueous layer to extract the desired compounds. The organic layers were combined and dried over magnesium sulfate and concentrated to give 45 mg of 2-10 as a white solid, 73%. mp 101° C. ¹H NMR δ 7.72 (1H, s), 7.54 (2H, m), 7.46 (2H, m), 7.38 (1H, m), 4.63 (2H, q, J=7.2 Hz), 4.04 (1H, s), 1.42 (3H, t, J=7.2 Hz); ¹³C NMR δ 155.76, 148.54, 141.16, 137.02, 128.80, 128.30, 125.51, 125.47, 68.73, 16.12. For the other analogues, the yields varied from 50-91%.

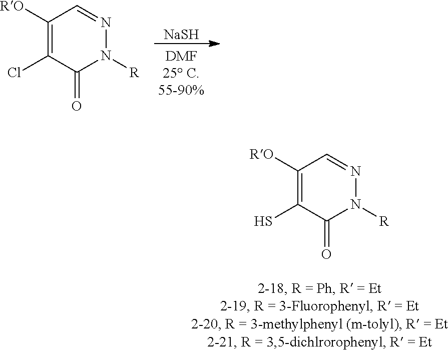

### EXAMPLE 7

### General Procedure for the Synthesis of 2-Substituted 5-Alkoxy-4-mercapto-pyridazin-3-ones

The procedures for 2-18 to 2-21 are the same as that of 2-10 with the corresponding starting materials. Yields: 45% to 85%.

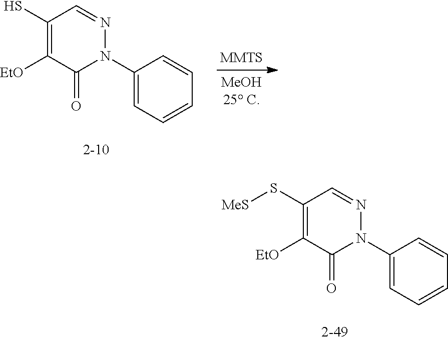

### EXAMPLE 8

### 4-Ethoxy-5-(methyldithio)-2-phenylpyridazin-3-one, 2-49

To a solution of 2-10 (6 mg, 0.024 mmol) in 2 mL of MeOH was added methyl methanethiosulfonate (MMTS, 4.5 mg, 0.036 mmol) at 25° C. The solution was stirred for 30 min and concentrated in vacuo. The residual mixture was subjected to flash column chromatography on silica gel to give 6.1 mg of 2-49, 88%. ¹H NMR δ 8.26 (1H, s), 7.57 (2H, m), 7.48 (2H, m), 7.40 (1H, m), 4.63 (2H, q, J=7.0 Hz), 2.52 (3H, s), 1.40 (3H, t, J=7.0 Hz); ¹³C NMR δ 155.42, 150.01, 141.15, 134.82, 128.69, 128.21, 127.79, 125.36, 68.78, 23.42, 15.85.

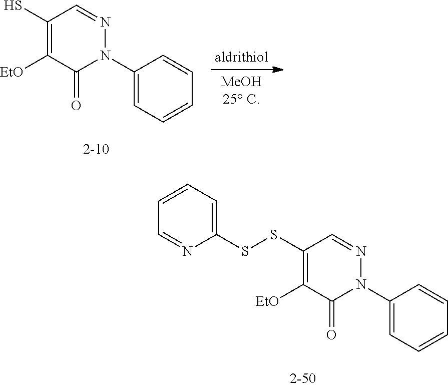

### EXAMPLE 9

### 4-Ethoxy-5-(2-pyridyldithio)-2-phenylpyridazin-3-one, 2-50

To a solution of 2-10 (6 mg, 0.024 mmol) in 2 mL of MeOH was added aldrithiol (7.9 mg, 0.036 mmol) at 25° C. The solution was stirred for 2 h and concentrated. The residual mixture was subjected to flash column chromatography on silica gel to give 5.6 mg of 2-50, 65%. ¹H NMR δ 8.51 (1H, d, J=4.0 Hz), 8.08 (1H, s), 7.68 (1H, ddd, J=8.0, 8.0, 1.5 Hz), 7.61 (1H, d, J=8.0 Hz), 7.54 (2H, m), 7.47 (2H, m), 7.38 (1H, m), 7.16 (1H, ddd, J=7.0, 5.0, 1.0 Hz), 4.70 (2H, q, J=7.0 Hz), 1.45 (3H, t, J=7.0 Hz); ¹³C NMR δ 157.60, 155.42, 150.51, 149.97, 141.06, 137.36, 135.34, 128.65, 128.22, 126.80, 125.29, 121.55, 120.30, 69.04, 15.91

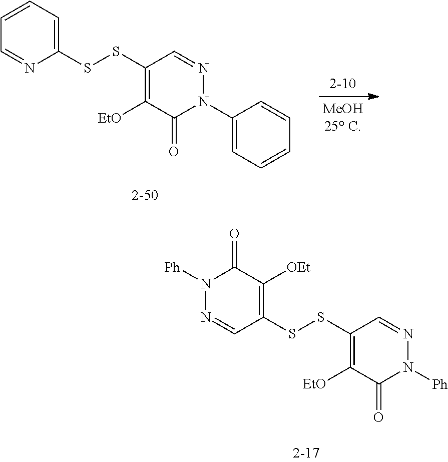

### EXAMPLE 10

### Bis(4-ethoxy-2-phenyl-5-pyridazyl)disulfide, 2-17

To a solution of 2-50 (10 mg, 0.028 mmol) in 2 mL of MeOH was added 15 mg of 2-10 at 25° C. The solution was stirred for 3 h then concentrated and subjected to flash column chromatography on silica gel to give 11.9 mg of 2-17, 85%. ¹H NMR δ 8.13 (1H, s), 7.55 (2H, m), 7.48 (2H, m), 7.39 (1H, m), 4.73 (2H, q, J=7.2 Hz), 1.43 (3H, t, J=7.2 Hz); ¹³C NMR(DMSO) δ 155.36, 150.61, 141.44, 136.57, 128.97, 128.57, 126.09, 121.58, 68.81, 16.03.

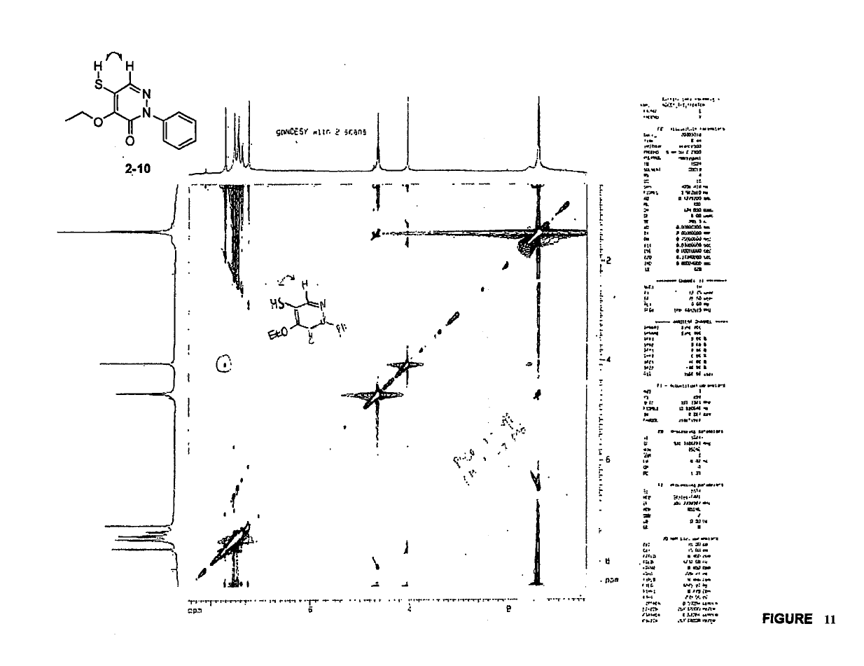

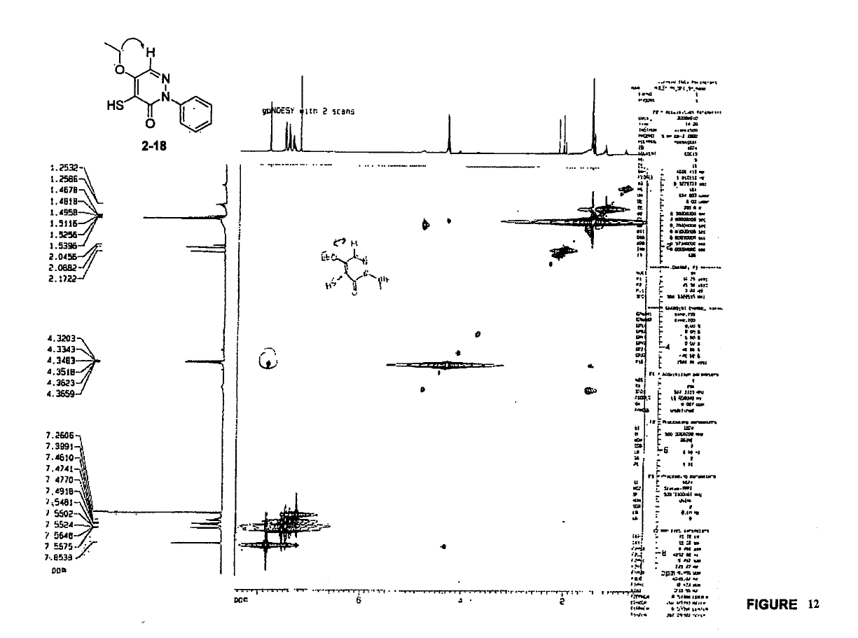

Additional information and the spectral data on specific compounds is included in the Tables (e.g., observed melting points are disclosed in Table 4) and Figures (e.g., one dimensional nuclear magnetic resonance (1D-NMR) data are disclosed in FIGS. 11-51, and two-dimensional nuclear magnetic resonance (2D-NMR) data are disclosed in FIGS. 52 and 53).

### EXAMPLE 11

### High-Throughput Screening

A total of 30,000 chemical compounds (DiverSet Chemically Diverse Library and Combichem Library, ChemBridge Corp.) were screened for S. aureus SrtA_{ΔN59} (residues 60 to 206) inhibition using an automated robotic system at the UCLA Molecular Screening Shared Resource facility. A fluorescence resonance energy transfer (FRET) assay was used in high-throughput screening in multi-well plates (384 wells per plate) (Suree, N.; Liew, C. K.; Villareal, V. A.; Thieu, W.; Fadeev, E. A.; Clemens, J. J.; Jung, M. E.; Clubb, R. T. 2009, (J. Biol. Chem. 2009, 284, 24465-24477). The assay monitors the SrtA_{ΔN59}-catalyzed hydrolysis of an internally quenched fluorescent substrate analogue (o-aminobenzoyl (Abz)-LPETG-diaminopropionic acid-dinitrophenyl-NH₂ (Dap(Dnp)), SynPep Corp. Dublin, Calif.) (Huang, X.; Aulabaugh, A.; Ding, W.; Kapoor, B.; Alksne, L.; Tabei, K.; Ellestad, G. Biochemistry 2003, 42, 11307). Briefly, 20 μL of purified SrtA (>95% homogeneity and proper folding was confirmed by 1D ¹H-NMR, final assay concentration of 0.4 μM in FRET buffer: 20 mM HEPES, 5 mM CaCl₂, 0.05% v/v Tween-20, pH 7.5) was incubated with 0.5 μL of test compound solution (dissolved in Me₂SO, final assay concentration of 10 μM) for 1 hour at 25° C. 32 wells of each plate were dedicated to positive and negative controls (1 μL of Me₂SO or 2 mM p-Hydroxymercuribenzoic acid was added alternatively to the test compound solution). Subsequently, 30 μL of fluorescent substrate solution (15 μM final assay concentration in FRET buffer) was added to the mixture to initiate the catalysis. Final Me₂SO concentrations were less than 2% in all assay mixtures. The FRET assays were monitored by a Flex Station II plate reader (Molecular Devices) with an excitation and emission wavelengths of 335 nm and 420 nm, respectively. The assay mixture was measured again after 5 hours for end-point reading.

### EXAMPLE 12

### Secondary Assays

For the top ten lead compounds, the concentration that is required for a 50% reduction in enzymatic activity (IC₅₀) was determined using well established methods (Kim, S. W.; Chang, I. M.; Oh, K. B. Biosci. Biotechnol. Biochem. 2002, 66, 2751; Copeland, A. R. Evaluation of Enzyme Inhibitors in Drug Discoveries; John Wiley & Sons: New Jersey, 2005; Huang, X.; Aulabaugh, A.; Ding, W.; Kapoor, B.; Alksne, L.; Tabei, K.; Ellestad, G. Biochemistry 2003, 42, 11307). Briefly, 20 μL of purified SrtA (final assay concentration of 1.5-15 μM in FRET buffer: 20 mM HEPES, 5 mM CaCl₂, pH 7.5) was incubated with 1 μL of test compound solution (dissolved in Me₂SO, final assay concentration of 0.08-400 μM) for 1 hour at 25° C. Subsequently, 30 μL of substrate solution in FRET buffer (37.5 μM final assay concentration for ^{Sa}SrtA, and 100 μM for ^{Ba}SrtA) was added to the mixture and the fluorescence was then monitored as described above. IC₅₀ values were calculated by fitting three independent sets of data to equation 1:

$$\frac{{v}_{i}}{{v}_{0}} = \frac{1}{1 + {( [ I ] / {IC}_{50} )}^{h}} \qquad ( Eq . 1 )$$

where vᵢ and v₀ are initial velocity of the reaction in the presence and absence of inhibitor at concentration [I], respectively. The term h is Hill coefficient.⁴⁶

Some of the inhibitors tightly bind to the enzyme such that their IC₅₀ values are lower than the enzyme concentration used in the assay (1.5-15 μM). To accurately define their potency the IC₅₀ values of these compounds were measured at different enzyme concentrations (Copeland, A. R. Evaluation of Enzyme Inhibitors in Drug Discoveries; John Wiley & Sons: New Jersey, 2005). If a linear relationship between total enzyme concentration [E]_{T} and IC₅₀ values was observed, the apparent dissociation constant for the enzyme-inhibitor (Kᵢ^{app}) was calculated by fitting the data to Morrison's quadratic equation (Eq. 2) (Williams, J. W.; Morrison, J. F. Methods Enzymol. 1979, 63, 437; Morrison, J. F. Biochim. Biophys. Acta 1969, 185, 269).

$$\frac{{v}_{i}}{{v}_{0}} = 1 - \frac{( {[ E ]}_{T} + [ I ] + {K}_{i}^{app} ) - \sqrt{{( {[ E ]}_{T} + [ I ] + {K}_{i}^{app} )}^{2} - {4 [ E ]}_{T} [ I ]}}{{2 [ E ]}_{T}} \qquad ( Eq . 2 )$$

### EXAMPLE 13

### Inhibitory Binding Reversibility Study

The reversibility of inhibition was determined by measuring the recovery of enzymatic activity after a sudden large dilution of the enzyme-inhibitor complex (Copeland, A. R. Evaluation of Enzyme Inhibitors in Drug Discoveries; John Wiley & Sons: New Jersey, 2005). 11.25 μL of purified SrtA at a concentration of 150 μM was mixed with 1.25 μL of each inhibitor such that the final inhibitor concentration was 10-fold greater than its IC₅₀. After incubation at 25° C. for 1 hour, 737.5 μL of FRET buffer was added. 30 μL of the diluted enzyme-inhibitor mixture was then plated and 20 μL of the fluorescent substrate (37.5 μM stock concentration) was added to initiate the cleavage reaction. The reaction progress curve was monitored as described above. Recovery of enzymatic activity after rapid dilution (100-fold) was calculated by comparing these progress curves with measurements of the reaction performed in the absence of inhibitor.

### EXAMPLE 14

### Mass Spectrometry

30 μL of purified SrtA (1.5 μM final assay concentration, dissolved in 5 mM CaCl₂, 20 mM HEPES, pH 7.5 buffer) was incubated with 1 μL of inhibitor such that the final inhibitor concentration was 1- and 10-fold higher than its IC₅₀ value. After incubating for 1, 48, or 96 hours at 25° C., the enzyme-inhibitor mixture was mixed with an equal amount of α-cyano-4-hydroxycinnamic acid, and analyzed by MALDI-TOF using a Voyager-DE STR Biospectrometry Workstation (Applied Biosystems). An equal amount (1 μL) of DMSO was used instead of the inhibitor solution as a negative control. Cbz-LPAT* (where Cbz is a carbobenzyloxy protecting group and T* is a threonine derivative that replaces the carbonyl group with —CH₂—SH) was used as a positive control, as it readily forms a disulfide bridge with the Cys184 thiol group of the enzyme (Jung, M. E.; Clemens, J. J.; Suree, N.; Liew, C. K.; Pilpa, R.; Campbell, D. O.; Clubb, R. T. Bioorg. Med. Chem. Lett. 2005, 15, 5076; Liew, C. K.; Smith, B. T.; Pilpa, R.; Suree, N.; Ilangovan, U.; Connolly, K. M.; Jung, M. E.; Clubb, R. T. FEBS Lett. 2004, 571, 221).

### EXAMPLE 15

### Determination of S. Aureus MIC

The minimal inhibitory concentration (MIC) was determined using the microtiter broth dilution method (Frankel, B. A.; Bentley, M.; Kruger, R. G.; McCafferty, D. G. J. Am. Chem. Soc. 2004, 126, 3404). An overnight saturated culture of S. aureus strain Newman (provided by Dr. Lloyd Miller, Division of Dermatology, David Geffen School of Medicine, UCLA) was diluted to an OD₆₀₀ of 0.01. After additional incubation at 37° C. and dilution to an OD₆₀₀ of 0.005, 180 μL of the culture was plated into a 96 well plate. 20 μL of inhibitor solution at varied concentrations (final concentrations of 0.1-100 μM) was then added to the culture. Cell growth was monitored by measuring the OD₆₀₀ during an overnight growth at 37° C. using a temperature-controlled plate reader. The cell growth percentage was calculated relative to cultures grown in the absence of inhibitor as well as in the presence of 10 μg/mL erythromycin. MIC measurements were performed in triplicate.

### EXAMPLE 16

### Molecular Docking

Molecular docking of each inhibitor was performed using Schrödinger Suite 2008 (Schrödinger Suite 2008; Schrödinger, LLC: New York, N.Y., USA) with an Induced-Fit Docking (IFD) workflow (Sherman, W.; Day, T.; Jacobson, M. P.; Friesner, R. A.; Farid, R. J. Med. Chem. 2006, 49, 534; Sherman, W.; Beard, H. S.; Farid, R. Chem. Biol. Drug Des. 2006, 67, 83). Calculations were run on a PC equipped with 3.8 GHz Intel Hyperthreading CPU, 2.0 GB SDRAM memory, and a LINUX operating system. The IFD protocol can be summarized as follows. First, the Glide docking module scales the van der Waals radii for both ligand and receptor binding site atoms by 50%. Second, the Prime module restores, predicts, and energy minimizes 20 structures of the given ligand-receptor complex generated by the first step. Finally, the ligand conformations are redocked into the induced-fit receptor structures generated by the second step. Complex structures possessing -energies that are within 30 kcal/mol were then ranked and the IFD scores determined. The poses presented in the paper are those conformations with the best score. The receptor protein structure was prepared by the Protein Preparation Wizard in Maestro user interface (Schrödinger, LLC) (Schrödinger Suite 2008; Schrödinger, LLC: New York, N.Y., USA). The bond orders were assigned, and the charges and hydrogen bonds were optimized by using the default protocol. All inhibitor ligands were prepared by the LigPrep (Schrödinger Suite 2008; Schrödinger, LLC: New York, N.Y., USA) module in a comparable manner.

### EXAMPLE 17

### Rationally Designed Dihydrooxazole Inhibitor

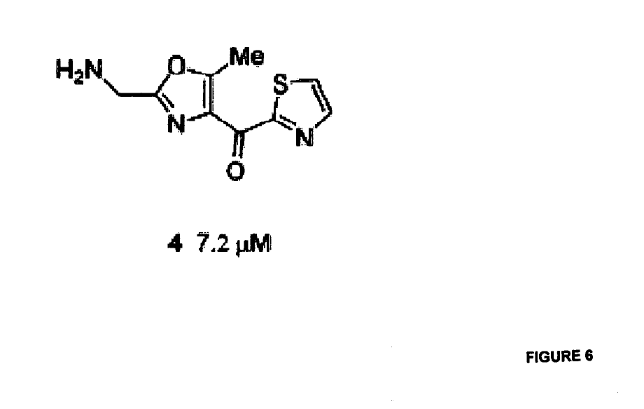

Synthesis of a ‘rationally designed’ inhibitor (compound 4). We designed and produced compound 4 (FIG. 6), which is a mechanism based inhibitor of SrtA. The IC₅₀ value of the compound 4 is 7.2 μM.

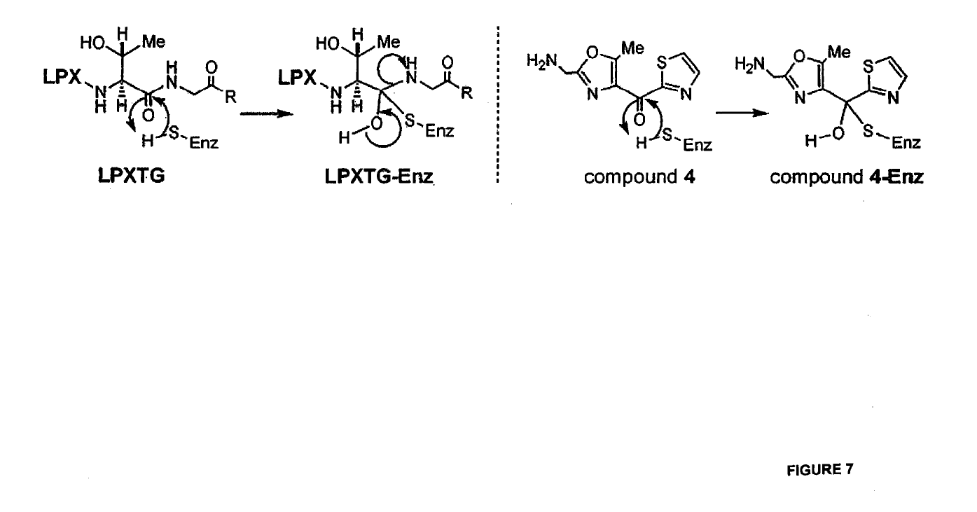

FIG. 7 illustrates one possible mechanism of how inhibition of SrtA is achieved. During normal catalysis the enzyme Cys184 thiol attacks the threonine carbonyl of the sorting signal to generate the first tetrahedral intermediate. Compound 4 is a smaller dihydrooxazole cyclic analogue of the sorting signal that, like the substrate, is attacked by the enzyme thiol to give the product 4-Enz. However, the thiazolyl ketone moiety of compound 4 stabilizes the tetrahedral complex and thus 4-Enz is relatively long lived. Importantly, this cyclic analog should also exhibit improved thiol selectivity as compared to conventional halomethyl ketone based inhibitors.

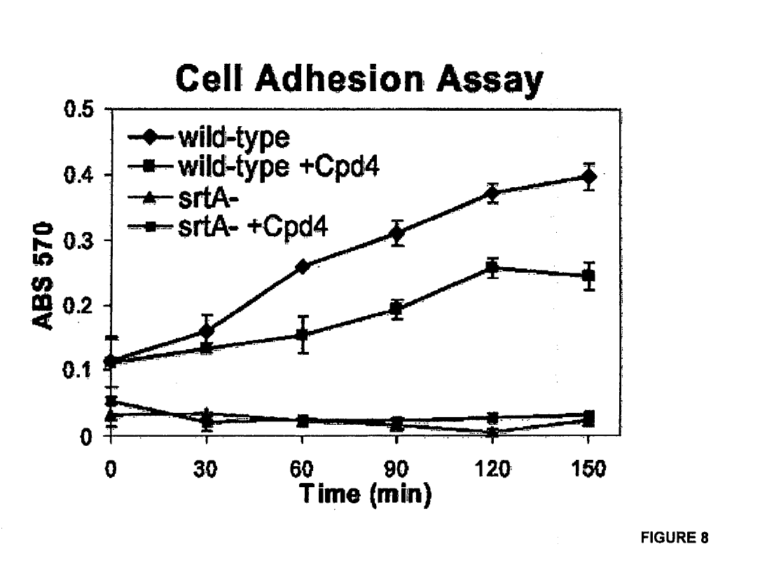

Biological activity. We have used two assays to show that compound 4 is a good inhibitor of SrtA. First, we have determined that it has an IC₅₀ value of 7.2 micromolar against the enzyme. Second, we implemented a cell adhesion assay that measures SrtA activity in vivo (FIG. 8). The assay works by monitoring whole cell adhesion to IgG coated plates, which is dependent on SrtA activity. Briefly, S. aureus strain RN4220 (wild-type) is grown at 37° C. to an OD₆₀₀ of 0.3. 1 mL aliquots of the culture are then removed every half hour for a period of 2.5 hours. The cells in each aliquot are washed by repeated centrifugation and resuspension in PBS buffer. The resuspended cells are then assayed for the presence of IgG-binding protein on their surfaces by applying them to a flat-bottom 96-well microtiter plate (Maxisorp surface, Nunc) that has been coated with 50 μg/mL of human IgG (Calbiochem). After repeated washing, the bacteria are fixed to the plates by the addition of 25% formaldehyde and stained with crystal violet to quantify the number of adhered cells by measuring the absorbance at 570 nm using a microplate reader (Molecular Devices, Spectramax M5). FIG. 8 shows that the assay readily discriminates between RN4220 (wild-type) and SKMI (SrtA−) strains of S. aureus. This data also shows that compound 4 inhibits protein display by SrtA in vivo.

### EXAMPLE 18

### General Procedure 1: Fisher Esterification of Amino Acids

Thionyl chloride (3 eq.) was added dropwise to a stirring solution of methanol at 0° C. in a flame dried round bottom flask equipped with a condenser followed by the amino acid (1 eq.) in one portion. The reaction mixture was then heated to reflux for 3 h, cooled to room temperature, concentrated in vacuo and thoroughly dried on the vacuum pump. Triethylamine (3 eq.) was then added to the crude HCl salt and the observed precipitate (triethylamine hydrochloride) was recrystallized from ethanol/ether. The triethylamine hydrochloride was filtered and washed with cold ethanol/ether (1:1). The filtrate was then concentrated and ample time was allowed in vacuo to remove excess triethylamine affording the crude product as the free base which was used without further purification.

### EXAMPLE 19

### General Procedure 2: N-Cbz Protection of Amino Acids

To a stirring solution of the amine (1 eq.) in H₂O/dioxane (4:1) was added NaOH (4 eq.) in one portion and the resulting solution was stirred 20 min. Benzyl chloroformate (1.5 eq.) was then added dropwise and the resulting solution was stirred for 12 h. The reaction mixture was then carefully acidified to pH=2 by addition of 1N HCl and extracted with ethyl acetate (3×). The organic phase was then dried over magnesium sulfate and concentrated in vacuo to the crude product which was either crystallized or used without further purification.

### EXAMPLE 20

### General Procedure 3: PyBOP Coupling

To a stirring solution of the carboxylic acid (1 eq.) in dichloromethane was added diisopropylethylamine (1 eq.) followed by PyBOP (1 eq.). After 5 min of stirring, the amine (1 eq.) was added and stirring was continued for 4 h. The reaction mixture was then diluted with ethyl acetate and washed with sat. aq. sodium bicarbonate (3×), sat. aq. ammonium chloride (3×), and finally brine (1×). The organic layer was then dried over magnesium sulfate, filtered, and the solvent was removed under reduced pressure to give the crude product which was purified by flash chromatography.

### EXAMPLE 21

### General Procedure 4: Dess-Martin Periodinane Oxidation of Alcohols to Ketones

To a stirring solution of the alcohol (1 eq.) in dichloromethane was added the Dess-Martin Periodinane reagent (1.4 eq.) and the resulting reaction mixture was stirred 1 h at room temperature. The reaction mixture was then filtered through a pad of Celite eluting with dichloromethane and the filtrate was concentrated to give the crude product which was purified by crystallization and/or column chromatography.

### EXAMPLE 22

### General Procedure 5: Saponification of Esters

To the ester (1 eq.) stirring in 3:1 THF/methanol was added an aqueous solution of 1M NaOH (2.5 eq.) under an inert atmosphere and stirring was continued 1 h or as judged by TLC. The solution was then adjusted to pH about 7 by the slow addition of 10% HCl solution and the residual THF and methanol were removed in vacuo without heating. The solution was then adjusted to pH=2 by the slow addition of 10% HCl solution and the resulting aqueous solution was extracted with ethyl acetate (3×). The organic layers were combined, dried over magnesium sulfate, and concentrated in vacuo to the crude products which were used in the ensuing steps without further purification.

### EXAMPLE 23

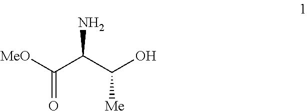

(2S,3R) 2-Amino-3-hydroxybutanoic acid methyl ester (1). This compound was prepared from L-threonine by the method described in General Procedure 1. Crude product crystallized on standing at 0° C. and was used without further purification. Pale yellow needles, R_{f}=0.22 (SiO₂, 8:2 CHCl₃/methanol). ¹H NMR (500 MHz, CDCl₃) δ 3.69 (m, 1H), 3.47 (s, 3H), 3.02 (bd, 1H, J=3.8 Hz), 2.51 (v bs, 3H), 0.94 (d, 3H, J=6.4 Hz); ¹³C NMR δ 174.1, 67.6, 59.5, 51.5, 19.4; MS (APCI) m/z 134 [M+H]⁺.

### EXAMPLE 24

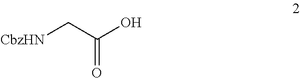

(Benzyloxycarbonylamino)acetic acid (2). This compound was prepared from L-glycine by the method described in General Procedure 2. After the workup described in the general procedure, the crude product was redissolved in ethyl acetate and washed with sat. aq. NaHCO₃ (3×). The combined aqueous phases were acidified to pH=2 with conc. HCl and then extracted with ethyl acetate (3×). The combined organic phases were dried over sodium sulfate, filtered and concentrated in vacuo to a white solid. White solid, 81% yield, R_{f} =0 (SiO₂, 9:1 hexanes/ethyl acetate). ¹H NMR (500 MHz, CD₃OD) δ 7.33 (m, 5H), 5.08 (s, 2H), 3.83 (s, 2H); ¹³C NMR δ 173.6, 159.0, 138.1, 129.4, 129.0, 128.8, 67.7, 43.1; MS (APCI) m/z=210 [M+H]⁺.

### EXAMPLE 25

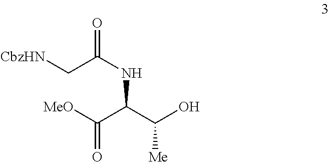

(2S,3R) 2-[(2-Benzyloxycarbonylamino)acetylamino]-3-hydroxybutanoic acid methyl ester (3). This compound was prepared by coupling 1 and 2 according to the method described in General Procedure 3. White solid, 75% yield, R_{f}=0.32 (SiO₂, 7:3 CH₂Cl₂/acetone). ¹H NMR (500 MHz, CD₃OD) δ 7.85 (d, 2H, J=8.8 Hz), 7.25-7.35 (m, 6H), 5.09 (s, 2H), 4.50 (dd, 1H, J=8.8, 2.9 Hz), 4.28 (m, 1H), 3.90 (s, 2H), 3.70 (s, 3H), 1.15 (d, 3H, J=6.4 Hz); ¹³C NMR δ 172.6, 172.3, 158.8, 137.8, 129.4, 128.9, 128.7, 68.2, 67.7, 59.1, 52.8, 44.7, 20.2; MS (APCI) m/z=325 [M+H]⁺.

### EXAMPLE 26

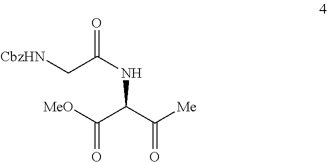

(2S)-2-[(2-Benzyloxycarbonylamino)acetylamino]-3-oxobutanoic acid methyl ester (4). This compound was prepared from 3 according to the method described in General Procedure 4. The product was purified by column chromatography followed by crystallization from ether/CHCl₃. White solid, 69% yield, R_{f}=0.17 (SiO₂, 9:1 CHCl₃/acetone). ¹H NMR (400 MHz, CDCl₃) δ 7.36 (m, 5H), 7.11 (bs, 1H), 5.42 (bs, 1H), 5.25 (d, 1H, J=6.4 Hz), 5.14 (s, 2H), 3.97 (d, 2H, J=5.5 Hz), 3.81 (s, 3H), 2.38 (s, 3H); ¹³C NMR δ 200.3, 172.0, 168.2, 156.2, 138.1, 129.5, 128.8, 67.9, 53.5, 52.3, 44.6, 27.7; MS (EI) m/z=322 [M+H]⁺.

### EXAMPLE 27

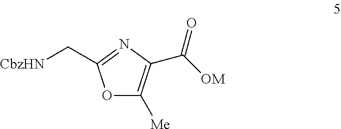

2-[(Benzyloxycarbonylamino)methyl]-5-methyloxazole-4-carboxylic acid methyl ester (5). To a stirring solution of triphenylphosphine (2.01 eq.), iodine (2 eq.) and triethylamine (4.01 eq.) in CH₂Cl₂ in a flame dried round bottom flask at room temperature was added 4 (1 eq.) as a solution in CH₂Cl₂. The reaction mixture was stirred 15 min then concentrated in vacuo without the use of heat to a wet brown solid. The wet solid was dissolved in sat. aq. Na₂S₂O₅, ether and a small amount of CHCl₃ (for solubility) and transferred to a separatory funnel. The aqueous layer was removed and the organic phase was washed with sat. aq. Na₂CO₃ (1×) then dried over magnesium sulfate, filtered and concentrated in vacuo to an amber solid which was purified by column chromatography. Beige solid, 78% yield, R_{f}=0.41 (SiO₂, 6:4 ethyl acetate/hexanes). ¹H NMR (500 MHz, CDCl₃) δ 7.21 (m, 5H), 6.06 (bt, 1H), 5.03 (s, 2H), 4.38 (d, 2H, J=5.6 Hz), 3.76 (s, 3H), 2.48 (s, 3H); ¹³C NMR δ 162.1, 158.9, 156.4, 156.0, 135.9, 128.1, 127.8, 127.7, 126.9, 66.7, 51.5, 37.8, 11.5; MS (EI) m/z=304 [M+H]⁺.

### EXAMPLE 28

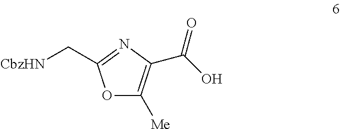

2-{(Benzyloxycarbonylamino)methyl]-5-methyloxazole-4-carboxylic acid (6). This compound was prepared from 5 using the method described in General Procedure 5. White solid, 98% yield, R_{f}=0 (SiO₂, 6:4 ethyl acetate/hexanes). ¹H NMR (500 MHz, d⁶-DMSO) δ 12.84 (v bs, 1H), 7.96 (bt, 1H), 7.35 (m, 5H), 5.04 (s, 2H), 4.27 (d, 2H, J=6.0 Hz), 2.52 (s, 3H); ¹³C NMR δ 162.9, 156.4, 156.2, 156.0, 136.8, 133.9, 128.3, 127.8, 127.7, 65.7, 39.5, 11.7; MS (MALDI) m/z=313 [M+Na]⁺.

### EXAMPLE 29

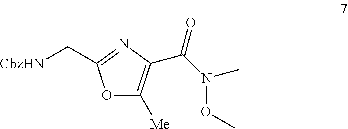

[4-(N-Methoxy-N-methylcarbamoyl)-5-methyloxazol-2-ylmethyl]carbamic acid benzyl ester (7). To a stirring solution of 6 (1 eq.) in THF in a flame-dried round bottom flask at 0° C. was added triethylamine (1 eq.) followed by ethyl chloroformate (1 eq.) as a solution in THF. The solution was allowed to warm to room temperature and after 0.5 h N,O-dimethylhydroxylamine hydrochloride (1 eq.) was added and stirring was continued for 16 h at room temperature. Additional ethyl chloroformate was added (0.5 eq.) followed by additional triethylamine (1 eq.) and stirring was continued 1 h at which time TLC indicated reaction completion. The reaction mixture was then concentrated in vacuo to a heterogeneous syrup which was dissolved in chloroform and water. The layers were separated and the aqueous layer was washed with chloroform (2×). The organic phases were combined, dried over magnesium sulfate, filtered and concentrated in vacuo to a white solid which was purified by flash chromatography. White solid, 87% yield, R_{f}=0.33 (SiO₂, 6:4 ethyl acetate/hexanes). ¹H NMR (500 MHz, CHCl₃) δ 7.32 (m, 5H), 5.46 (bt, 1H), 5.13 (s, 2H), 4.46 (d, 2H, J=5.5 Hz), 3.75 (s, 3H), 3.35 (s, 3H), 2.50 (s, 3H); ¹³C NMR δ 157.5, 156.1, 155.0, 154.9, 136.1, 129.0, 128.5, 128.2, 128.1, 67.2, 61.6, 38.3, 11.8; MS (MALDI) m/z=356 [M+Na]⁺.

### EXAMPLE 30

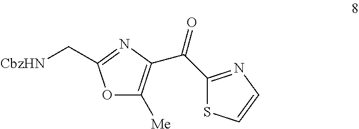

[5-Methyl-4-(thiazole-2-carbonyl)-oxazol-2-ylmethyl]carbamic acid benzyl ester (8). To a stirring solution of n-BuLi (1.6M in hexanes, 1.3 eq.) in ether in a flame-dried round bottom flask at −78° C. was added a solution of freshly distilled 2-bromothiazole (2 eq.) in ether dropwise so as not to increase the temperature of the reaction. The resulting solution was stirred at −78° C. for 0.5 h and then a solution of 7 (1 eq.) in ether was slowly added so as not to increase the temperature of the reaction mixture and on completion of addition, the mixture was stirred 30 min during which time it retained a light beige color. The reaction was quenched with sat. aq. NaHCO₃ which turned the reaction mixture to a very dark brown color. The mixture was warmed to room temperature over 15 min, diluted with sat. aq. NaHCO₃ and washed with ethyl acetate (3×). The organic phases were combined, dried over magnesium sulfate, filtered and concentrated in vacuo to a beige oil which was purified by flash chromatography. Beige oil, 74% yield, R_{f}=0.40 (SiO₂, 92.5:7.5 CHCl₃/acetone). ¹H NMR (500 MHz, CHCl₃) δ 8.11 (d, 1H, J=3.0 Hz), 7.67 (d, 1H, J=2.5 Hz), 7.33 (m, 5H), 5.64 (bt, 1H), 5.14 (s, 2H), 4.57 (d, 2H, J=6.0 Hz), 2.68 (s, 3H); ¹³C NMR δ 177.3, 164.7, 159.1, 158.6, 156.2, 145.0, 136.1, 132.9, 128.5, 128.2, 128.1, 126.3, 67.2, 38.3, 12.8; MS (EI) m/z=357 [M+H]⁺.

### EXAMPLE 31

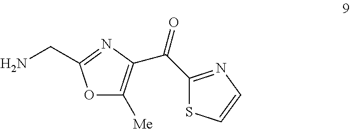

(2-Aminomethyl-5-methyl-oxazol-4-yl)-thiazol-2-yl-methanone (9). To a stirring solution of 8 (1 eq.) in CH₂Cl₂ in a flame-dried round bottom flask at room temperature was added a 33% solution HBr in acetic acid (40 eq. HBr) all at once and the resulting solution was stirred for 15 min then concentrated in vacuo without using heat. Water was added and the resulting solution was washed with hexanes (3×) and the organic phases were discarded. The aqueous layer was brought to pH=9-10 by addition of concentrated aq. NH₄OH and was then washed with CH₂Cl₂ (3×). The combined organic phases were dried over magnesium sulfate, filtered and concentrated in vacuo to a yellow solid which was purified by flash chromatography. Bright yellow solid, quant. yield, R_{f}=0.42 (SiO₂, 9:1 CHCl₃/methanol). ¹H NMR (500 MHz, CHCl₃) δ 8.07 (d, 1H, J=2.9 Hz), 7.65 (d, 1H, J=2.9 Hz), 3.94 (s, 2H), 2.63 (s, 3H), 1.67 (bs, 2H); ¹³C NMR δ 177.4, 164.9, 162.5, 158.7, 144.9, 132.6, 126.1, 39.2, 12.6; MS (MALDI) m/z=224 [M+H]⁺.

### EXAMPLE 32

Additional compounds Several derivatives of the pyridazinone series that have even better activity than many of the compounds discussed above are disclosed herein. Four of these compounds are potent sortase inhibitors (2-58, 2-59, 2-60 and 2-61). The structures and measured inhibitory properties of the compounds 2-58, 2-59, 2-60, and 2-61 are shown in Table 4. All of the compounds inhibit the SrtA sortase enzyme from Staphylococcus aureus with sub-micromolar IC₅₀ values. They are therefore the most potent sortase inhibitors that have ever been reported. This data further substantiates that molecules with a pyridazinone scaffold are potent sortase inhibitors.

General procedures for the synthesis of compounds such as compounds 2-58, 2-59, 2-60, and 2-61 are discussed in the following Examples.

### EXAMPLE 33

### General Procedure for the Synthesis of YJ-08E Series

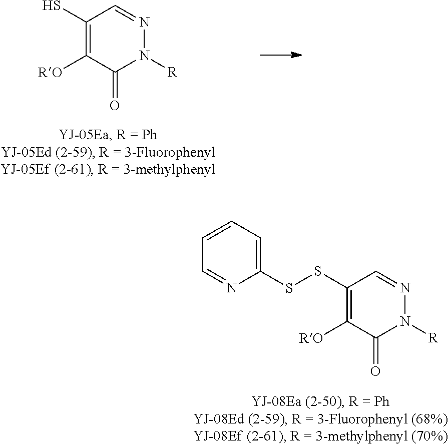

To a solution of YJ-05Ea (6 mg, 0.024 mmol) in 2 mL of methanol was added Aldrithiol (7.9 mg, 0.036 mmol) at 25° C. The solution was stirred for 2 h at room temperature and concentrated in vacuo. The residual mixture was subjected to flash column chromatography to give 5.6 mg of YJ-08Ea, 65%.

4-Ethoxy-2-(3-fluorophenyl)-5-(pyridin-2-yldisulfanyl)pyridazin-3(2H)-one, YJ-08Ed (2-59). ¹H NMR δ 8.51 (1H, bd, J=5.0 Hz), 8.09 (1H, s), 7.68 (1H, td, J=7.8, 1.7 Hz), 7.58 (1H, bd, J=8.0 Hz), 7.40 (2H, m), 7.35 (1H, bd, J=10.0 Hz) 7.17 (1H, ddd, J=7.5, 5, 1 Hz), 7.08 (1H, m), 4.69 (2H, q, J=7 Hz), 1.44 (3H, t, J=7 Hz).

4-Ethoxy-5-(pyridin-2-yldisulfanyl)-2-3-methylphenylpyridazin-3(2H)-one, YJ-08Ef (2-61). ¹H NMR δ 8.50 (1H, bd, J=5 Hz), 8.06 (1H, s), 7.67 (1H, td, J=7.5, 2.0 Hz), 7.60 (1H, bd, J=8.0 Hz), 7.32 (3H, m), 7.17 (2H, m), 4.70 (2H, q, J=7 Hz), 2.38 (3H, s) 1.44 (3H, t, J=7 Hz). ¹³C NMR δ 157.73, 155.57, 150.63, 150.18, 141.11, 138.85, 137.48, 135.35, 129.19, 128.62, 126.87, 126.02, 122.54, 121.66, 120.41, 69.14, 21.37, 16.03

### EXAMPLE 34

### General Procedure for the Synthesis of YJ-09E Series

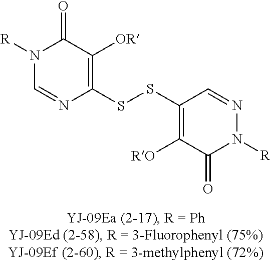

To a solution of YJ-08Ea (10 mg, 0.028 mmol) in 2 mL of methanol was added 15 mg of YJ-05Ea at 25° C. The solution was stirred for 3 hours then concentrated in vacuo and subjected to flash column chromatography to give 11.9 mg of YJ-O9Ea, 85%.

5,5′-Disulfanediylbis(4-ethoxy-2-(3-fluorophenyl)pyridazin-3(2H)-one), YJ-09Ed (2-58). ¹H NMR δ 8.13 (1H, s), 7.40 (3H, m), 7.11 (1H, m), 4.73 (2H, q, J=7.25 Hz), 1.41 (3H, t, J=7.25 Hz)

4-Ethoxy-5-((5-ethoxy-6-oxo-1-3-methylphenyl-1,6-dihydropyridazin-4-yl)disulfanyl)-2-3-methylphenylpyridazin-3(2H)-one, YJ-09Ef (2-60). ¹H NMR δ 8.11 (1H, s), 7.34 (3H, m), 7.21 (1H, bd, J=7.0 Hz), 4.73 (2H, q, J=7.0 Hz), 2.44 (3H, s) 1.41 (3H, t, J=7 Hz)

### EXAMPLE 35

### The Inhibitors Disrupt Sortase Mediated Protein Anchoring to the Cell Wall

The majority of sortase inhibitors reported to date have only been shown to inhibit the enzymatic activity of the purified enzyme. However, in order for a compound to be an effective anti-infective agent it must be able to specifically inhibit sortase mediated protein attachment to the cell wall in intact bacterial cells. We therefore developed a cell-based approach to monitor sortase activity and employed it to verify the cellular efficacy of our compounds (manuscript in preparation). The assay monitors the activity of the sortase A enzyme from Bacillus anthracis, which like the Staphylococcus aureus enzyme is inhibited by our compounds in vitro (Bioorganic & Medicinal Chemistry 17 2009; p 7174-85). Below, I briefly describe the new cell-based assay and new data generated using the assay that demonstrates that our compounds inhibit sortase mediated protein anchoring.

Assay: A B. subtilis strain expressing the B. anthracis sortase A enzyme and a cellulase reporter enzyme was constructed. 15 mL cultures were inoculated with this strain and grown to an A₆₀₀ of 0.05. The inhibitors were then added to the cultures and incubated for 20 minutes prior to the addition of xylose to induce SrtA expression. When the cells reached an A₆₀₀ of 0.1, IPTG was added to induce expression of cellulase reporter enzyme. After 2 hours of cellulase expression, 3 mL samples were collected, washed and resuspended in 0.5% carboxymethylcellulose (CMC) to measure cellulase activity. CMC hydrolysis continued for 1 hour, after which the cells were pelleted, and the supernatant was analyzed for glucose release using dinitrosalicylic acid. The appropriate controls were performed and cellulase activity was rigorously shown to be dependent upon sortase activity (data not shown).

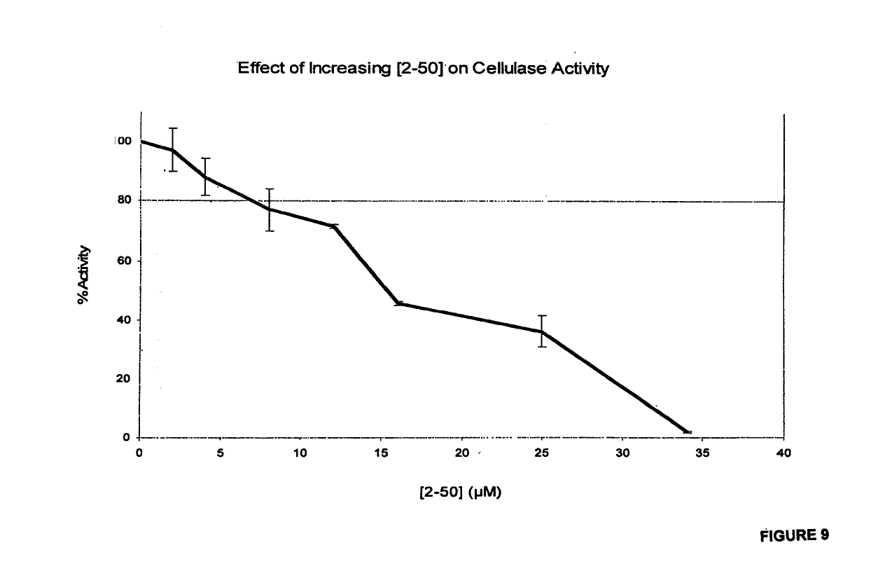

Assay results: A detailed analysis of compound 2-50 is shown in FIG. 9. It shows a plot of cellulase activity as a function of inhibitor concentration in the bacterial culture. The activity is a measure of the amount of functional cellulase enzyme anchored to the cell wall by the sortase enzyme. As can be seen from the data, sortase activity is inhibited in a dose-dependent manner by the progressive addition of 2-50. Near complete inhibition occurs about 34 μM compound. This indicates that the ability of sortase to display surface proteins is inhibited by compound 2-50. From this data the EC₅₀ value of compound 2-50 is about 15 μM. Importantly, the EC₅₀ is generally similar to the IC₅₀ value of the compound against the isolated enzyme.

A similar test was performed using compounds: 2-50, 2-59, 3-12 and 3-17. However, in this assay only a single concentration of the compound was tested. The concentration used for each molecule was 20-times its previously determined IC₅₀ value (Bioorganic & Medicinal Chemistry 17 2009; p 7174-85). For each, the sortase activity in cell culture was determined by measuring cellulase activity and the numbers were normalized to values obtained for cell cultures in which no inhibitor had been added. FIG. 2 shows that at these compound concentrations about 30-40% of sortase activity is inhibited. From this data the EC₅₀ of the molecules is estimated be slightly larger than 8, 8, 28 and 34 μM for compounds 2-50, 2-59, 3-12 and 3-17, respectively.

In total, the compounds and compositions disclosed herein provide molecules that inhibit the ability of sortase to attach proteins to the cell wall. As cell wall attached proteins play an important role in processes that promote bacterial pathogenesis in S. aureus and other pathogens, it is believed that these compounds have potent anti-infective properties.

1. A pyridazinone compound having the structure: 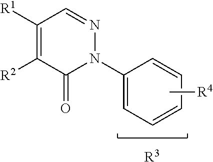
Wherein:
R1 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;
R2 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;
R3 is alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and, where R3 is phenyl or cyclohexyl, and then the pyridazinone compound has five R4 substituents, wherein R4 is independently hydrogen, hydroxyl, halogen, nitroxyl, alkyl, alkenyl, alkynyl, acyl, aryl, cycloalkyl, cycloaryl, haloalkyl, alkyl-substituted aryl, alkyl substituted cyclohexyl, halogen-substituted aryl, or halogen-substituted cyclohexyl, alkyloxy, or aryloxy,
with the proviso that compounds named herein 2(lead), 2-1, 2-2, 2-5 to 2-10, 2-22, 2-25, 2-27, 2-28, 2-39 and 2-42 to 2-48 are excluded.

2-3. (canceled)

4. A compound of claim 1 having the structure: 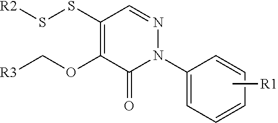
Wherein
Five R1 substituents are independently hydrogen, hydroxyl, halogen, nitroxyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;
R2 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy; and
R3 is hydrogen, hydroxyl, halogen, nitroxyl, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy.

5. A compound of claim 1 selected from the compounds named herein 2-3, 2-11, 2-12, 2-13, 2-14, 2-15, 2-16, 2-17, 2-18, 2-19, 2-20, 2-21, 2-23, 2-24, 2-26, 2-29, 2-30, 2-31, 2-32, 2-33, 2-34, 2-35, 2-36, 2-37, 2-38, 2-40, 2-41, 2-49 and 2-50.

6. A compound of claim 1 selected from

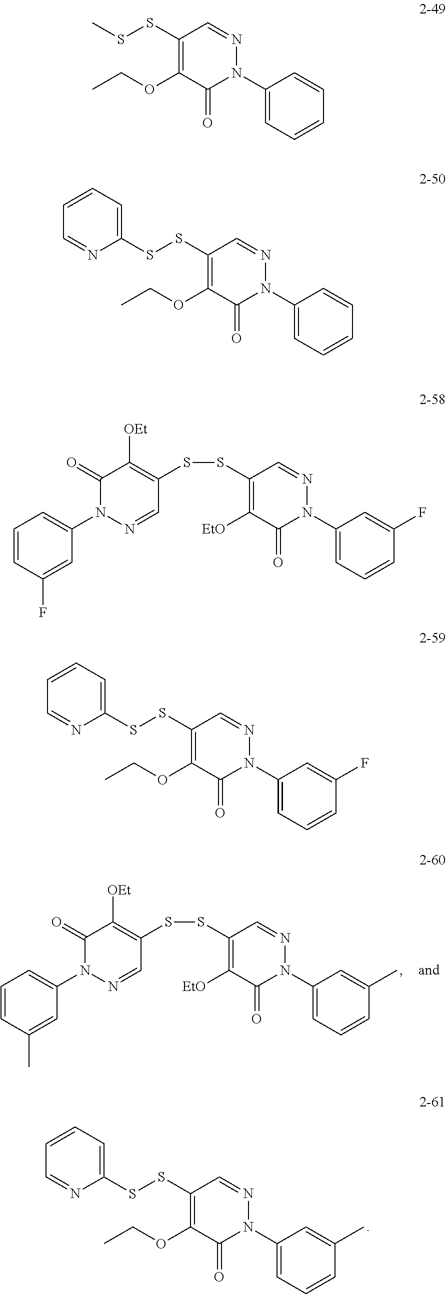

7-9. (canceled)

10. A pyrazolethione or pyrazolone compound having the structure: 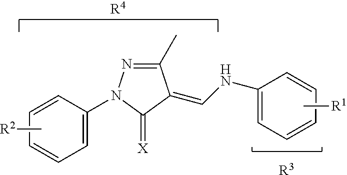
Wherein
X is O or S;
Five R1 substituents are independently hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;
R2 is hydrogen, hydroxyl, halogen, sulfhydryl, sulfoxyl, substituted sulfyl, alkyl, alkenyl, alkynyl, acyl, aryl, haloalkyl, cycloalkyl, cycloaryl, alkyl-substituted aryl, alkyl substituted cyclohexyl, halogen-substituted aryl, or halogen-substituted cyclohexyl, alkyloxy, or aryloxy;
R3 is cyclohexyl, cycloaryl, substituted cycloaryl, substituted cyclohexyl, pyridinyl, alkyl-substituted aryl, alkyl substituted cyclohexyl, halogen-substituted aryl, or halogen-substituted cyclohexyl; and
R4 includes any suitable R2 and X,
with the proviso that compounds named herein 3(lead), 3-1, 3-2, 3-3, 3-4, 3-5, 3-6, 3-7, 3-8, 3-9, 3-10, 3-11, 3-12, 3-13, 3-14, 3-15, 3-16, 3-17, 3-18, 3-19, 3-20, and 3-21 are excluded.

11. A compound selected from:

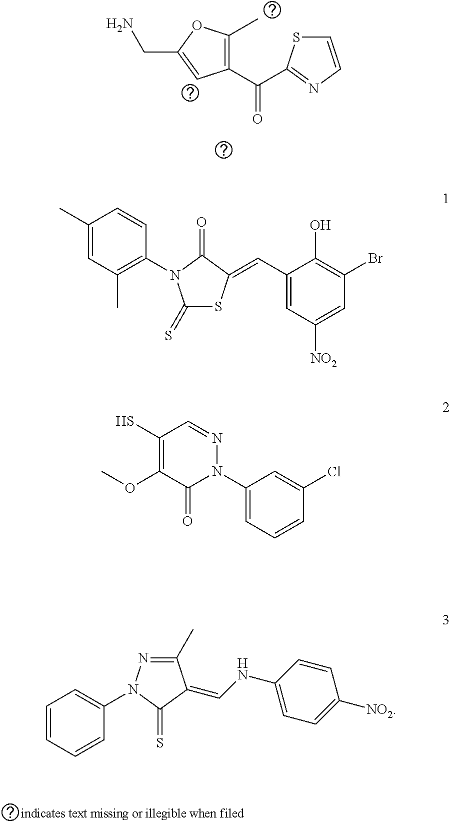

12. (canceled)

13. A pharmaceutical composition comprising an effective amount of a compound of claim 1, in admixture with a pharmaceutically acceptable carrier.

14-15. (canceled)

16. A pharmaceutical composition comprising an effective amount of a pyridazinone compound of claim 6 in admixture with a pharmaceutically acceptable carrier.

17-26. (canceled)

27. A method of treating a subject in need of treatment, comprising administering an effective dose of a pharmaceutical composition of claim 13.

28-36. (canceled)

37. A pharmaceutical composition comprising an effective amount of a compound of claim 10, in admixture with a pharmaceutically acceptable carrier.

37. A pharmaceutical composition comprising an effective amount of a compound of claim 11, in admixture with a pharmaceutically acceptable carrier.
